(19) United States
(12) Patent Application Publication
(10) Pub. No.: US 2024/0189078 A1
(43) Pub. Date: Jun. 13, 2024

(54) RESTORATIVE DECISION SUPPORT FOR DENTAL TREATMENT

(71) Applicant: Align Technology, Inc., San Jose, CA (US)

(72) Inventors: Christopher E. Cramer, Durham, NC (US); Michael Austin Brown, Orlando, FL (US); Magdalena Blankenburg, Berlin (DE); Shipra Jain, Zürich (CH); Alexander Okupnik, Zürich (CH)

(21) Appl. No.: 18/530,984

(22) Filed: Dec. 6, 2023

Related U.S. Application Data
(60) Provisional application No. 63/431,434, filed on Dec. 9, 2022.

(51) Int. Cl. A61C 13/00; A61B 1/00; A61B 1/24; A61B 6/00; A61B 6/03; A61B 6/51; A61C 7/00; G06T 7/00; G06T 7/50; G06T 7/62; G06T 7/70; G06V 10/764; G06V 10/82; G16H 30/20

(52) U.S. Cl. CPC A61C 13/0004; A61B 1/000096; A61B 1/24; A61B 6/032; A61B 6/51; A61B 6/5247; A61C 7/002; G06T 7/0012; G06T 7/50; G06T 7/62; G06T 7/70; G06V 10/764; G06V 10/82; G16H 30/20; G06T 2200/24; G06T 2207/20081; G06T 2207/20084; G06T 2207/30036; G06V 2201/03

(57) ABSTRACT

A method of providing restorative decision support for a dental patient includes receiving image data of an intraoral cavity of a patient, the image data corresponding to one or more imaging modalities; deriving a plurality of parameters from the image data; applying a decision model to the plurality of parameters; and generating a restorative decision recommendation based on an output of the decision model.

### RELATED APPLICATION(S)

The present application claims the benefit under 35 U.S.C. § 119(e) of U.S. Provisional Patent Application No. 63/431,434, filed Dec. 9, 2022, which is hereby incorporated by reference herein in its entirety.

### TECHNICAL FIELD

Embodiments of the present disclosure relate to the field of dental diagnostics and, in particular, to a system and method for improving the process of providing restorative decisions.

### BACKGROUND

For a typical dental practice, a patient visits the dentist at least twice a year for a cleaning and an examination. A dental office may or may not generate a set of x-ray images of the patient's teeth during the patient visit. The dental hygienist additionally cleans the patient's teeth and notes any possible problem areas, which they convey to the dentist. The dentist then reviews the patient history, reviews the new x-rays (if any such x-rays were generated), and spends a few minutes examining the patient's teeth and gums in a patient examination process. During the patient examination process, the dentist may follow a checklist of different areas to review. The examination can start with examining the patient's teeth for cavities, then reviewing existing restorations, then checking the patient's gums, then checking the patients' head, neck and mouth for pathologies or tumors, then checking the jaw joint, then checking the occlusion and bite relationship and/or other orthodontic problems, and then checking any x-rays of the patient. Based on this review, the dentist makes a determination as to whether there are any dental conditions that need to be dealt with immediately and whether there are any other dental conditions that are not urgent but that should be dealt with eventually and/or that should be monitored. The dentist then needs to explain the identified dental conditions to the patient, talk to the patient about risks, benefits, potential treatments or restorations, alternatives, and consequences of no treatment, and motivate the patient to make an informed decision on treatment for their health.

A goal of dentistry is to maintain as much of a patient's natural teeth as possible. Where a tooth has been damaged or has decay, a restoration is indicated to prevent fracturing of the tooth which could lead to a root canal or extraction. Restorations are broadly divided into two categories: (i) direct restorations and (ii) indirect restorations. Direct restorations involve drilling out the damaged or decayed natural tooth material and applying a “filling” made of amalgam, composite, gutta pertcha, gold foil, temporary filling or other material. Indirect restorations may be used when a tooth is damaged or decayed and there is no longer enough natural tooth material to support a direct restoration. Indirect restorations include crowns, bridges, inlays, onlays, and veneers. Full coverage restorations (crowns or caps) are used when the structural integrity of the tooth's cusps have been compromised, because teeth tend to fracture when the cusps are weakened. If a cusp/tooth fractures, then a tooth may require a root canal/core build-up/crown or, in the worst case, extraction.

Current practice is for each doctor to rely on their clinical judgement to make decisions on the restoration type. Frequently, however, patients are skeptical of a doctor's recommendation for a crown or other restoration, and may assume the doctor is making the recommendation primarily from a profit motive, or the patient may not understand the consequences of no treatment because the doctor was not able to successfully communicate the need for treatment. Consequently, patients may refuse treatments or restorations that are in their best interest despite objective evidence supporting the doctor's recommendations.

### SUMMARY

Multiple example implementations are summarized. Many other implementations are also envisioned.

In a first implementation, a method of providing restorative decision support for a dental patient comprises: receiving image data of an intraoral cavity of a patient, the image data corresponding to one or more imaging modalities; deriving a plurality of parameters from the image data; applying a decision model to the plurality of parameters; and generating a restorative decision recommendation based on an output of the decision model.

In at least one embodiment, the one or more imaging modalities comprises an imaging modality selected from an intraoral scan, a radiograph, or a cone-beam computed tomography (CBCT) scan.

In at least one embodiment, the one or more imaging modalities comprises the intraoral scan, and wherein the image data comprises one or more three-dimensional (3D) point clouds and at least one of two-dimensional (2D) near infrared (NIR) images, 2D ultraviolet images, or 2D color images.

In at least one embodiment, the one or more imaging modalities comprise the radiograph, and wherein the image data comprises one or more of a panoramic radiograph, a bitewing radiograph, or a periapical radiograph.

In at least one embodiment, the image data corresponds to two or more of the imaging modalities.

In at least one embodiment, the plurality of parameters are each selected from a geometric parameter, a volume/area parameter, or a fracture classification parameter.

In at least one embodiment, the geometric parameter comprises an inter-cuspal width.

In at least one embodiment, the volume/area parameter comprises one or more of a restorative volume proportion, a decay volume proportion, or a restorative surface proportion.

In at least one embodiment, the fracture classification parameter comprises information descriptive of a tooth fracture location and a tooth fracture depth.

In at least one embodiment, the decision model comprises one or more of a decision tree or a neural network.

In at least one embodiment, the restorative decision recommendation comprises one or more of: a direct restoration recommendation or an indirect restoration recommendation, or an indication of a dental condition and a severity level for the dental condition.

In at least one embodiment, the dental condition is selected from a group consisting of caries, gum recession, tooth wear, malocclusion, tooth crowding, tooth spacing, plaque, tooth stains, tooth cracks, cervical defects, and chipped or broken teeth.

In at least one embodiment, the method further comprises: presenting the restorative decision recommendation for display in a graphical user interface (GUI).

In at least one embodiment, the method further comprises: storing, in a record associated with a dentist to whom the restorative decision recommendation was provided, the restorative decision recommendation and an actual restorative decision made by the dentist in a key performance indicator (KPI) database.

In a second implementation, a method comprises: identifying a tooth having an associated dental condition based on a first set of parameters derived from current image data of an intraoral cavity of a patient; presenting in a user interface (UI), such as a graphical user interface (GUI) a 2D or 3D image of the intraoral cavity of the patient and an indication of the tooth having the associated dental condition; and presenting in the GUI a restorative decision recommendation based on an output of a decision model for which the first set of parameters is used as input.

In at least one embodiment, the restorative decision recommendation is presented in the GUI responsive to a user selection of the tooth in the 2D or 3D image, and wherein the indication comprises one or more of a label on the tooth, an outline over the tooth, or a color of the tooth.

In at least one embodiment, identifying the tooth having the associated dental condition comprises: comparing the first set of parameters to a second set of parameters derived from prior image data of the intraoral cavity captured prior to the current image data; and identifying the tooth by determining that a difference between a parameter from the first set of parameters and a parameter from the second set of parameters satisfies a threshold condition.

In at least one embodiment, the current image data corresponds to a first imaging modality, and wherein the prior image data corresponds to a second imaging modality that is different from the first imaging modality.

In a third implementation, a method comprises: receiving image data corresponding to an intraoral cavity of a patient; applying a trained machine learning model to the image data to derive a plurality of parameters from the image data; and applying a decision model to the plurality of parameters to generate a restorative decision recommendation.

In at least one embodiment, the trained machine learning model is adapted to compute or estimate a volume of restorative material present on or in a tooth in the image data, and wherein deriving the plurality of parameters comprises computing at least one of a restorative volume or a surface proportion from the estimated volume of restorative material.

In at least one embodiment, the trained machine learning model is adapted to receive image data corresponding to different imaging modalities as input, and wherein the different imaging modalities are independently selected from an intraoral scan, a radiograph, or a cone-beam computed tomography (CBCT) scan.

In a further implementation, a dental diagnostics system comprises a memory and a processing device to execute instructions from the memory to perform the method of any of the preceding implementations. For example, in at least one embodiment, the method comprises: receiving image data of an intraoral cavity of a patient, the image data corresponding to one or more imaging modalities; deriving a plurality of parameters from the image data; applying a decision model to the plurality of parameters; and generating a restorative decision recommendation based on an output of the decision model.

In a further implementation, an intraoral scanning system comprises an intraoral scanner and a computing device operatively connected to the intraoral scanner, wherein the computing device is to perform the method of any of the preceding implementations.

In a further implementation, a computer readable medium includes instructions that, when executed by a processing device, cause the processing device to perform the method of any of the preceding implementations.

### BRIEF DESCRIPTION OF THE DRAWINGS

The present disclosure is illustrated by way of example, and not by way of limitation, in the figures of the accompanying drawings.

FIG. 1A illustrates a user interface of a dental diagnostics hub, in accordance with at least one embodiment of the present disclosure.

FIG. 1B illustrates a user interface of a dental diagnostics hub, showing a time-lapse feature, in accordance with at least one embodiment of the present disclosure.

FIG. 1C illustrates a user interface of a dental diagnostics hub after diagnostics have been run on a patient's dental arches, in accordance with at least one embodiment of the present disclosure.

FIG. 1D illustrates a user interface for navigating diagnostics results provided to a mobile device by a dental diagnostics hub, in accordance with at least one embodiment of the present disclosure.

FIG. 1E illustrates a user interface of a dental diagnostics hub after diagnostics have been run on a patient's dental arches, in accordance with at least one embodiment of the present disclosure.

FIG. 2 illustrates a user interface for a caries analysis of a dental diagnostics hub, in accordance with at least one embodiment of the present disclosure.

FIG. 3 illustrates a user interface for a amalgam analysis of a dental diagnostics hub, in accordance with at least one embodiment of the present disclosure.

FIG. 4 illustrates an intraoral scanning system, in accordance with at least one embodiment of the present disclosure.

FIG. 5 illustrates a 3D model of a lower dental arch comparing a prediction to a labeled model to show the quality of the segmentation between the segmented and labeled restorative material, in accordance with at least one embodiment of the present disclosure.

FIG. 6 illustrates classification of tooth decay regions comparing a prediction to a labeled model, in accordance with at least one embodiment of the present disclosure.

FIG. 7 illustrates the segmentation of identified restorative material and an occlusal surface of a tooth, in accordance with at least one embodiment of the present disclosure.

FIG. 8 illustrates a user interface for a key performance indicator dashboard, in accordance with at least one embodiment of the present disclosure.

FIG. 9 illustrates a flow diagram for a method of generating a restorative decision recommendation, in accordance with at least one embodiment of the present disclosure.

FIG. 10 illustrates a flow diagram for a method of presenting a restorative decision recommendation in a dental diagnostics hub, in accordance with at least one embodiment of the present disclosure.

FIG. 11 illustrates a flow diagram for a method of generating a restorative decision recommendation from parameters derived from image data by a trained machine learning model, in accordance with at least one embodiment of the present disclosure.

FIG. 12 illustrates a block diagram of an example computing device, in accordance with at least one embodiment of the present disclosure.

### DETAILED DESCRIPTION

Described herein are embodiments of a method for providing restorative decision support, which may be implemented, for example, as part of a dental diagnostics hub. In at least one embodiment, a dentist or doctor (terms used interchangeably herein) and/or their technicians may gather various information about a patient. Such information may include image data corresponding to one or more imaging modalities, including, but not limited to, intraoral 3D scans of the patient's dental arches, x-rays of the patient's teeth (e.g., optionally including bitewing x-rays of the patient's teeth, panoramic x-rays of the patient's teeth, periapical and occlusal x-rays, etc.), cone-beam computed tomography (CBCT) scans of the patient's jaw, infrared images of the patient's teeth, or color 2D images of the patient's teeth and/or gums. Other information may include, but is not limited to, biopsy information, malocclusion information, observation notes about the patient's teeth and/or gums, and so on. The intraoral scans may be generated by an intraoral scanner, and at least some of the other data may be generated by one or more devices other than intraoral scanners. Additionally, different data may be gathered at different times. Each of the different data points may be useful for determining whether the patient has one or more types of dental conditions. The methodologies described herein may be used to derive a plurality of parameters from the image data (e.g., geometric parameters, volume/area parameter, or fracture classification parameters, each of which is discussed in greater detail below), and applying a decision model to the plurality of parameters in order to generate a restorative decision recommendation that the doctor may provide to the patient. The recommendation may be presented within the dental diagnostics hub, which may provide a user interface that presents a unified view of the restorative decision recommendation, each of the types of analyzed dental conditions, an indication of which of the types of dental conditions might be of concern, and which of the types of dental conditions might not be of concern for the patient.

Certain embodiments leverage one or more imaging modalities to support doctors in making a clinical decision regarding restoration. The imaging modalities may include, for example, images generated from intraoral scanners, radiographs (bitewing, periapical, or panoramic), CBCT scans, etc. The parameters derived from this information can be used by the restorative decision support system and methodologies to provide a customizable, objective measure of the health of a tooth, and provide a recommendation of either a direct restoration (e.g., a filling) or a particular indirect restoration (e.g., inlay, onlay, crown, bridge, or veneer). By providing objective evidence of the need for direct or indirect restoration, the restorative decision support system advantageously assists doctors in discussing restorative solutions with their patients, which can help both doctors and patients to make an informed decision and while reducing the likelihood of a tooth fracture that could lead to a root canal, core build-up, crown, or extraction. In at least one embodiment, a customizable decision tree model is implemented to support the clinical decision process for identifying a tooth requiring a restoration and helping to identify the restoration type.

In at least one embodiment, a restorative decision system can identify a tooth having an associated dental condition based on a set of parameters derived from image data of an intraoral cavity of a patient. A 2D or 3D image of the intraoral cavity can be presented in a graphical user interface (GUI), for example, of a diagnostics hub, along with an indication of the associated dental condition. The GUI can further present a restorative decision recommendation based on an output of a decision model for which the set of parameters are used as an input. The GUI further provides an option for the user (e.g., the doctor) to visualize the recommendation in response to a user selection of the tooth in the 2D or 3D image. For example, the tooth may be labeled, outlined, or colored, in order to draw attention to it.

In at least one embodiment, the restorative decision system can receive image data corresponding to an intraoral cavity of a patient (e.g., from multiple different imaging modalities). A trained machine learning model may be applied to the image data to derive a set of parameters from the image data. For example, the trained machine learning model can be adapted to compute or estimate a volume of restorative material present on or in a tooth in the image data (e.g., a 2D or 3D image). From such estimations, one or more parameters can be derived, such as a restorative volume or surface proportion. A decision model (e.g., a decision tree, a neural network, etc.) may be subsequently applied to these parameters to generate the restorative decision recommendation.

Traditionally, the various types of gathered information are fragmented such that each of the different types of information is stored in a separate system and accessed by a separate dentistry-related application. In order for a dentist to fully assess a patient's dental health, they generally would need to separately load and review each of the different types of data in each of the different dentistry-related applications specific to that type of data. The standard process for reviewing the data and making diagnoses involves numerous manual steps on the part of the dentist, and requires significant effort and time on the part of the dentist. Accordingly, the standard process for a dentist to perform a full analysis of a patient's dental health is highly inefficient. Moreover, once such a full analysis is made, it is generally difficult for the dentist to present the full analysis to the patient, which is again a highly manual process on the part of the dentist. What is lacking in traditional systems, and what is provided in embodiments described herein, is a way to quickly and consistently gather patient dental information in a systematic fashion, store the information in a central repository, and derive various parameters from such information that is useful in producing treatment and dental restoration recommendations.

In at least one embodiment, the dental diagnostics hub brings together all of the disparate types of information associated with a patient's dental health. The dental diagnostics hub further performs automated analysis for each of the different types of dental conditions. A summary result of the various automated analyses may then be shown together in a GUI. The summary result of the various automated analyses may include a severity rating for each of the types of dental conditions. The summary result may identify those dental conditions having higher severity levels to call them to the attention of the dentist. The dentist may then select any of the types of dental conditions to cause the dental diagnostics hub to provide more detailed information about the selected type of dental condition for the patient.

In at least one embodiment, the dental diagnostics hub greatly increases the speed and efficiency of diagnosing dental conditions of patients. The dental diagnostics hub enables a dentist to determine, at a single glance of the GUI for the dental diagnostics hub, all of the dental conditions that might be of concern for a patient. It enables the dentist to easily and quickly prioritize dental conditions to be addressed. Additionally, the dental diagnostics hub may compare different identified dental conditions to determine any correlations between different identified dental conditions. As a result, the dental diagnostics hub may identify some dental conditions as symptoms of other underlying root cause dental conditions. For example, the dental diagnostics hub may identify tooth crowding and caries formation that results from the tooth crowding. Additionally, the dental diagnostics hub in at least one embodiment creates presentations of dental conditions, what will happen if those dental conditions are untreated, root causes of the patient's dental conditions, treatment plan options, and/or simulations of treatment results. Such presentations may be shown to the patient to educate the patient about the condition of their dentition and their options for treating the problems and/or leaving the problems untreated.

A dental practitioner (e.g., a dentist or dental technician) may use an intraoral scanner to perform an intraoral scan of a patient's oral cavity. An intraoral scan application running on a computing device operatively connected to the intraoral scanner may communicate with the scanner to effectuate intraoral scanning and receive intraoral scan data (also referred to as intraoral images and intraoral scans). A result of the intraoral scanning may be a sequence of intraoral scans that have been discretely generated (e.g., by pressing on a “generate scan” button of the scanner for each image) or automatically generated (e.g., by pressing a “start scanning” button and moving the intraoral scanner around the oral cavity while multiple intraoral scans are generated). An operator may start performing intraoral scanning at a first position in the oral cavity, and move the intraoral scanner within the oral cavity to various additional positions until intraoral scans have been generated for an entirety of one or more dental arches or until a particular dental site is fully scanned. In at least one embodiment, recording of intraoral scans may start automatically as teeth are detected or insertion into the oral cavity is detected and may automatically be paused or stopped as removal of the intraoral scanner from the oral cavity is detected.

According to an example, a user (e.g., a dental practitioner) may subject a patient to intraoral scanning. In doing so, the user may apply the intraoral scanner to one or more patient intraoral locations. The scanning may be divided into one or more segments. As an example the segments may include a lower buccal region of the patient, a lower lingual region of the patient, a upper buccal region of the patient, an upper lingual region of the patient, one or more preparation teeth of the patient (e.g., teeth of the patient to which a dental device such as a crown or an orthodontic alignment device will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with scan being directed towards an interface area of the patient's upper and lower teeth). In at least one embodiment, the segments include an upper dental arch segment, a lower dental arch segment and a patient bite segment. Via such scanner application, the scanner may generate intraoral scan data. The computing device executing the intraoral scan application may receive and store the intraoral scan data.

The intraoral scan data may corresponding to different imaging modalities, which may include two-dimensional (2D) intraoral images (e.g., color 2D images), three-dimensional intraoral scans (e.g., intraoral images with depth information such as monochrome height maps), intraoral images generated using infrared or near-infrared (NIRI) light, and/or intraoral images generated using ultraviolet light. The 2D color images, 3D scans, NIRI and/or infrared images and/or ultraviolet images may be generated by an intraoral scanner capable of generating each of these types of intraoral scan data. Such intraoral scan data may be provided from the scanner to the computing device in the form of one or more points (e.g., one or more pixels and/or groups of pixels). For instance, the scanner may provide such intraoral scan data as one or more point clouds.

In at least one embodiment, intraoral scanning may be performed on a patient's oral cavity during a visitation of a dentist's office. The intraoral scanning may be performed, for example, as part of a semi-annual or annual dental health checkup. The intraoral scanning may be a full scan of the upper and lower dental arches, and may be performed in order to gather information for performing dental diagnostics. The dental information generated from the intraoral scanning may include 3D scan data, 2D color images, NIRI and/or infrared images, and/or ultraviolet images.

In addition to performing intraoral scanning, a dental practitioner may generate one or more other types of relevant dental health information, such as x-rays of the patient's teeth (e.g., optionally including bitewing x-rays of the patient's teeth, panoramic x-rays of the patient's teeth, etc.), cone-beam computed tomography (CBCT) scans of the patient's jaw, infrared images of the patient's teeth, color 2D images of the patient's teeth and/or gums not generated by an intraoral scanner (e.g., from photos taken by a camera), biopsy information, malocclusion information, observation notes about the patient's teeth and/or gums, and so on. For example, in addition to a dental practitioner generating an intraoral scan of the oral cavity during an annual or semi-annual dentist appointment, the dental practitioner may additionally generate one or more x-rays of the patient's oral cavity during the dentist appointment. Additional types of dental information may also be gathered when the dentist deems it appropriate to generate such additional information. For example, the dentist may take biopsy samples and send them to a lab for testing and/or may generate a panoramic x-ray and/or a CBCT scan of the patient's oral cavity.

The intraoral scan application may generate a 3D model (e.g., a virtual 3D model) of the upper and/or lower dental arches of the patient from the intraoral scan data. To generate the 3D model(s) of the dental arches, the intraoral scan application may register and stitch together the intraoral scans generated from the intraoral scan session. In at least one embodiment, performing image registration includes capturing 3D data of various points of a surface in multiple intraoral scans, and registering the intraoral scans by computing transformations between the intraoral scans. The intraoral scans may then be integrated into a common reference frame by applying appropriate transformations to points of each registered intraoral scan.

In at least one embodiment, registration is performed for each pair of adjacent or overlapping intraoral scans. Registration algorithms may be carried out to register two adjacent intraoral scans for example, which essentially involves determination of the transformations which align one intraoral scan with the other. Registration may involve identifying multiple points in each intraoral scan (e.g., point clouds) of a pair of intraoral scans, surface fitting to the points of each intraoral scans, and using local searches around points to match points of the two adjacent intraoral scans. For example, the intraoral scan application may match points, edges, curvature features, spin-point features, etc. of one intraoral scan with the closest points, edges, curvature features, spin-point features, etc. interpolated on the surface of the other intraoral scan, and iteratively minimize the distance between matched points. Registration may be repeated for each adjacent and/or overlapping scans to obtain transformations (e.g., rotations around one to three axes and translations within one to three planes) to a common reference frame. Using the determined transformations, the intraoral scan application may integrate the multiple intraoral scans into a first 3D model of the lower dental arch and a second 3D model of the upper dental arch. The intraoral scan data may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

The intraoral scan application or another application may further register data from one or more other imaging modalities to the 3D model generated from the intraoral scan data. For example, processing logic may register x-ray images, CBCT scan data, ultrasound images, panoramic x-ray images, 2D color images, NIRI images, and so on to the 3D model. Each of the different imaging modalities may contribute different information about the patient's dentition. For example, NIRI images and x-ray images may identify caries and color images may be used to add accurate color data to the 3D model, which is usable to determine tooth staining. The registered intraoral data from the multiple imaging modalities may be presented together in the 3D model and/or side-by-side with one or more imaging modalities shown that reflect a zoomed in and/or highlighted section and/or orientation of the 3D model. The data from different imaging modalities may be provided as different layers, where each layer may be for a particular imaging modality. This may enable a doctor to turn on or off specific layers to visualize the dental arch with or without information from those particular imaging modalities.

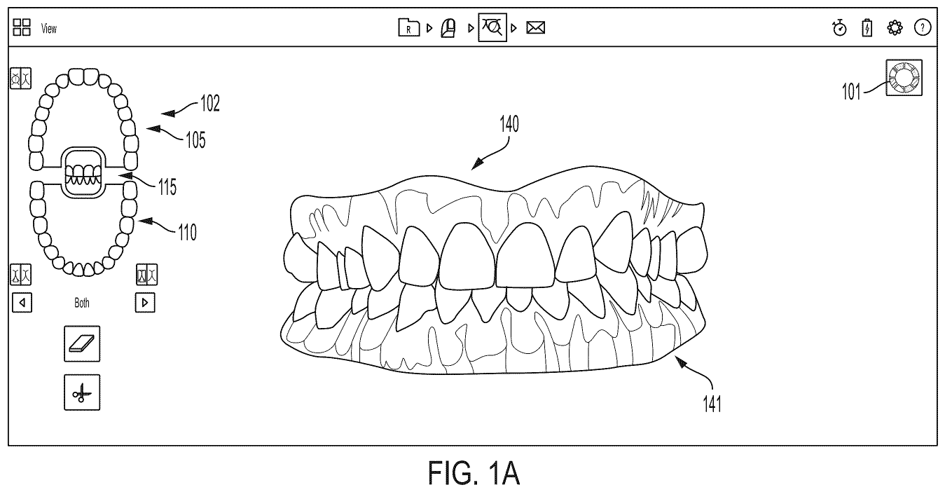

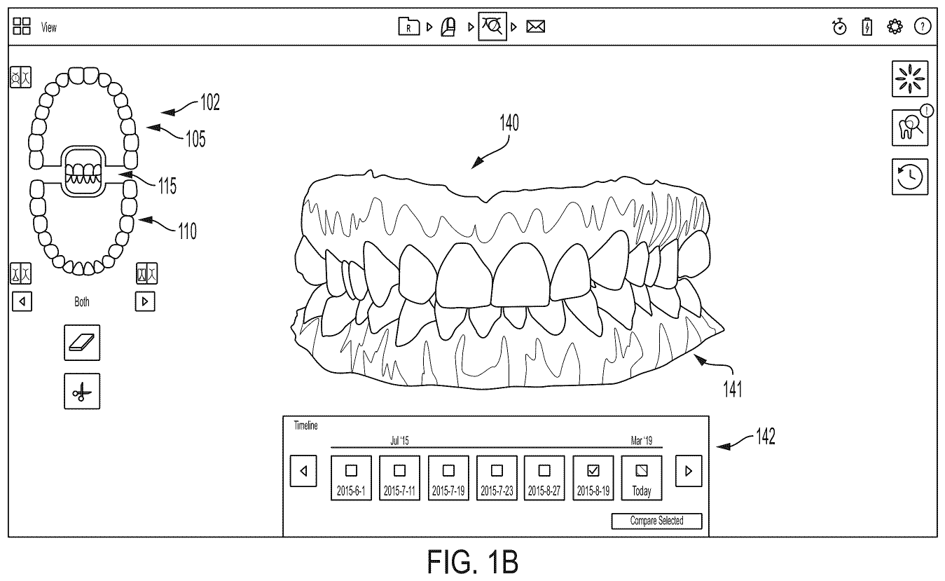

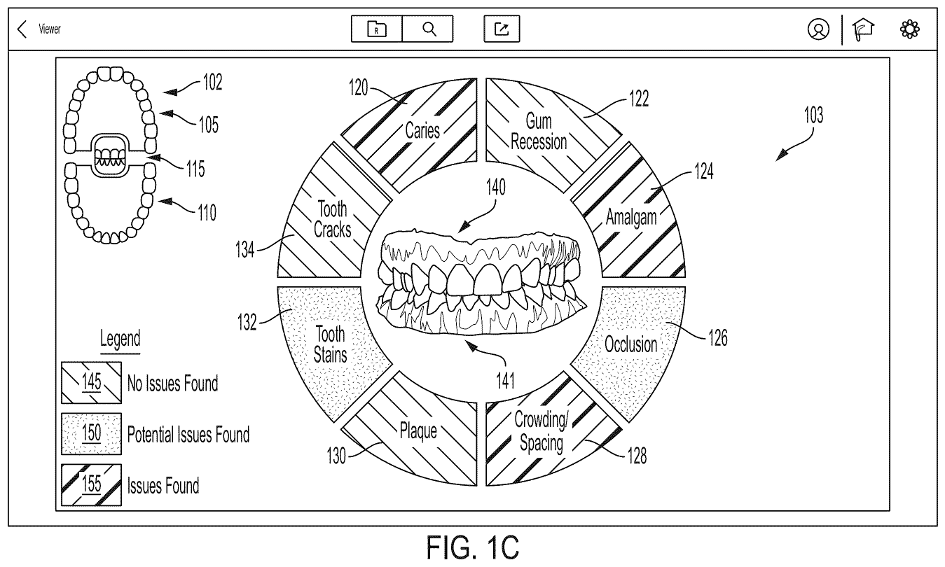

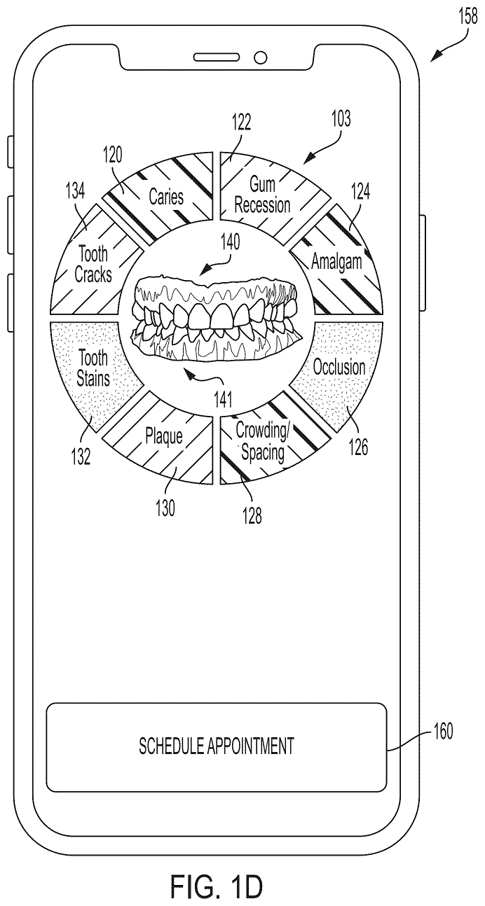

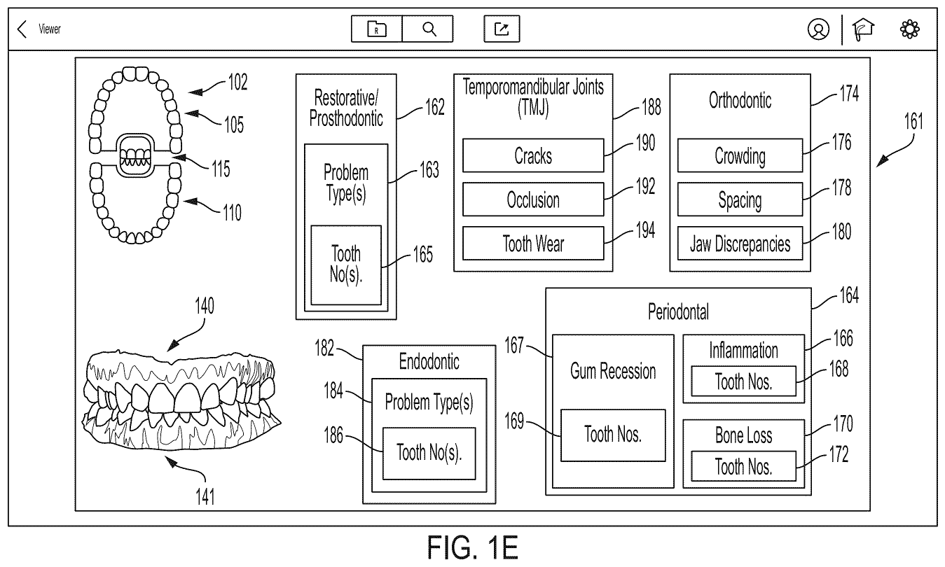

FIG. 1A illustrates a user interface of a dental diagnostics hub, in accordance with at least one embodiment of the present disclosure. A 3D model of a patient's upper dental arch 140 and a 3D model of the patient's lower dental arch 141 may be generated by an intraoral scan application and input into the dental diagnostics hub. Additionally, bite data showing the relationship of the upper dental arch 140 and the lower dental arch 141 may be input into the dental diagnostics hub. The bite relationship data may also include how the upper and lower jaw dynamically relate to each other in functional motions and not just in a static relationship relative to each other. This can be useful for diagnostic problems related to the jaw joint (e.g., temporomandibular (TMJ) disorders). Additionally, intraoral scans may have been generated of one or more preparation tooth of the patient, which may also be input to the dental diagnostics hub. The dental diagnostics hub may then present a view of the upper dental arch 140, the lower dental arch 141, the preparation teeth, and/or the relative positions of the upper and lower dental arches 140, 141 in the user interface of the dental diagnostics hub.

Via the user interface of the dental diagnostics hub, a practitioner may view one or more of the upper dental arch 140, the lower dental arch 141, a particular preparation tooth and/or the patient bite, each of which may be considered a separate scan segment or mode. The practitioner may select one or multiple scan segments to view via a scan segment selector 102. In at least one embodiment, as shown, the scan segment selector 102 may include an upper dental arch segment selection 105, a lower dental arch segment selection 110 and a bite segment selection 115. As illustrated, the upper dental arch segment selection 105 and the lower dental arch segment selection 110 are active, causing the 3D model of the upper dental arch 140 and the 3D model of the lower dental arch 141 to be shown. A practitioner may rotate the 3D models and/or change a zoom setting for a view of the 3D models using the GUI.

The GUI of the dental diagnostics hub may further include a diagnostics command 101. Selection of the diagnostics command 101 may cause the dental diagnostics hub to perform one or multiple different analyses of the patient's dental arches 140, 141 and/or bite. The analyses may include an analysis for identifying tooth cracks, an analysis for identifying gum recession, an analysis for identifying tooth wear, an analysis of the patient's occlusal contacts, an analysis for identifying crowding of teeth (and/or spacing of teeth) and/or other malocclusions, an analysis for identifying plaque, an analysis for identifying tooth stains, an analysis for identifying caries, and/or other analyses of the patient's dentition. Once the analyses are complete, a dental diagnostics summary may be generated and shown in the GUI of the dental diagnostics hub, as shown in FIG. 1C.

Some of the analyses that are performed to assess the patient's dental health are dental condition progression analyses that compare dental conditions of the patient at multiple different points in time. For example, one carries assessment analysis may include comparing caries at a first point in time and a second point in time to determine a change in severity of the caries between the two points in time, if any. Other time-based comparative analyses that may be performed include a time-based comparison of gum recession, a time-based comparison of tooth wear, a time-based comparison of tooth movement, a time-based comparison of tooth staining, and so on. In at least one embodiment, processing logic automatically selects data collected at different points in time to perform such time-based analyses. Alternatively, a user may manually select data from one or more points in time to use for performing such time-based analyses.

FIG. 1B illustrates a user interface of a dental diagnostics hub, showing a time-lapse feature 142, in accordance with at least one embodiment of the present disclosure. In at least one embodiment, the time-lapse feature is launched automatically when a user selects the diagnostics command 101 to provide the user an option to select which dental information from which points in time to use for the analyses to be performed. In at least one embodiment, the time-lapse feature 142 shows each of the different points in time (i.e., different times stamps) at which dental information was collected along a time line. The dental information that may be selected may include at a minimum intraoral scan data (e.g., 3D models generated based on one or more intraoral scanning sessions). The dental information that may be selected may further include x-rays generated at various points in time, CBCT scan data generated at various points in time, and/or other dental information generated at various points in time. Via the time-lapse feature 142, a user may select one or more past data points (e.g., for previously generated 3D models of dental arches) and/or one or more current data points or most recent data points (e.g., for a current 3D model of the dental arches). The selected data points may then be used to perform one or more time-based analyses of the patient's dentition.

In at least one embodiment, the time-based analyses of the patient's dentition compare 3D models and/or one or more dental conditions of the patient over time, and identify dental conditions and/or determine a rate of progression of the one or more dental conditions based on the comparison. For example, 3D models of the dental arches from different points in time may be compared to one another to determine rates of progression of tooth wear, caries development, gum recession, gum swelling, malocclusions, and so on. The rates of progression may be compared to rate of progression thresholds. The rate of progression thresholds may be set by a doctor or may be set to defaults. Amount of change for dental conditions may also be determined, and may be compared to amount of change thresholds. Those dental conditions for which the rate of progression meets or exceeds a rate of progression threshold for that dental condition and/or for which amount of change meets or exceeds an amount of change threshold may be identified as dental conditions that are of clinical significance and/or dental conditions for which issues or problems have been identified.

The time-based analyses may project detected rates of progression or rates of change of one or more dental conditions into the future to predict severity levels of the dental conditions at future points in time. In at least one embodiment, progression of one or more dental conditions may be projected into the future, and the predicted dental condition at each projected point in time may be compared to one or more criteria (e.g., such as a severity threshold). The one or more criteria may be default criteria and/or may be criteria set by a doctor (e.g., a user of the dental diagnostics hub). The criteria may also be set by aggregated data, either within the same practice or through a network of practices of similar patient traits. When the one or more criteria are satisfied, that indicates that a dental condition of clinical significance is identified. The future point in time at which a projected dental condition will satisfy the one or more criteria (e.g., pass the severity threshold) may be noted and added to the patient's record in at least one embodiment. In at least one embodiment, if the future point in time at which a projected dental condition will satisfy the one or more criteria for that dental condition is within a threshold amount of time from a current date, then the dental condition may be identified as of clinical importance or of potential clinical importance.

FIG. 1C illustrates a user interface of a dental diagnostics hub showing a dental diagnostics summary 103 after diagnostics have been run on a patient's dental arches, in accordance with at least one embodiment of the present disclosure. In at least one embodiment, the dental diagnostics summary 103 includes the scan segment selector 102 including upper dental arch segment selection 105, lower dental arch segment selection 110 and/or bite segment selection 115. In at least one embodiment, the dental diagnostics summary 103 further includes views of the selected dental segments or modes (e.g., of the 3D model of the upper dental arch 140 and the 3D model of the lower dental arch 141 of the patient).

The dental diagnostics summary 103 provides a single view showing multiple different types of possible dental conditions and assessments as to the presence and/or severity of each of the types of dental conditions. In at least one embodiment, the various dental conditions are assigned one of three severity levels, including “no issues found” 145, “potential issues found” 150 and “issues found” 155. Each of the dental conditions may be coded or labeled with the severity ranking determined for that type of dental condition. In at least one embodiment, the dental conditions are color coded to graphically show severity levels. For example, those dental conditions for which issues were found may be coded red, those dental conditions for which potential issues were found may be coded yellow, and those dental conditions for which no issues were found may be coded green. Many other coding schemes are also possible. In at least one embodiment, each of the dental conditions is assigned a numeric severity level. For example, on a scale of 1 to 100, each dental condition may be assigned a severity level between 1 and 100 to indicate the severity level of that dental condition. Those dental conditions with a severity level that is below a first threshold severity level may be identified as dental conditions for which no issues were found. Those dental conditions for which the severity level is above the first threshold severity level but below a second threshold severity level may be identified as dental conditions for which potential issues were found. Those dental conditions for which the severity level is above the second severity level threshold may be identified as dental conditions for which issues were found. In at least one embodiment, different severity level thresholds may be set for each of the different dental conditions. Alternatively, the severity levels of the different dental conditions may be normalized across the multiple types of dental conditions and the same severity level thresholds may be used for multiple dental conditions. In at least one embodiment, dental conditions are ranked based on their severity levels and/or based on the different between their severity levels and the associated severity level threshold for the dental conditions.

In at least one embodiment, a doctor may set severity level thresholds for one or more of the dental conditions. Severity level thresholds that a doctor may set may be point-in-time severity level thresholds for point-in-time severity levels of dental conditions determined based on data from a single point in time. Additionally, or alternatively, severity level thresholds that a doctor may set may be time-dependent thresholds, such as amount of change thresholds and rate of change thresholds. Alerts may be set to remind the doctor when the threshold level is approaching specific criteria.

Absent such selected severity level thresholds, default severity level thresholds may be automatically set for one or more of the dental conditions. In an example, a doctor may set a caries size threshold, and any detected caries that have a size that meets or exceeds the set caries size threshold may be identified as a found issue. In another example, a doctor may set a gum recession amount threshold, and any identified gum recession that has a value that meets or exceeds the gum recession amount threshold may be identified as a found issue. A doctor may also set rate of change thresholds for one or more dental conditions and/or such rate of change thresholds may be automatically set to default values. For example, if the rate of change of tooth wear exceeds a tooth wear rate of change threshold, then the patient may be identified as having an identified tooth wear issue. A doctor may also set an amount of change threshold. If a detected amount of change is greater than the set amount of change threshold for a dental condition, then the doctor may be alerted. Multiple different units may be used to set severity level thresholds, such as units of distance (e.g., microns, millimeters, fractions of an inch, etc.), units of size (e.g., microns, millimeters, fractions of an inch, etc.), units of rates of change (e.g., microns/month, millimeters per year, etc.), units of luminance, units of volume (e.g., mm³), units of area (e.g., mm²), ratios, percentages (e.g., percentage of change), and so on.

In at least one embodiment, severity level thresholds may depend at least in part on a location of an identified dental condition. For example, different caries severity thresholds may be set for different locations. Caries that are close to dentin may be more urgent because they are more likely to cause pain and/or to require a root canal than caries that are far from dentin. Accordingly, caries that are close to dentin may have a lower threshold than caries that are far from dentin, for example. In at least one embodiment, the distance between a caries and a patient's dentin may be determined based on x-ray data, a CBCT scan and/or NIRI imaging of the intraoral cavity.

In at least one embodiment, the different types of dental conditions for which analyses are performed and that are included in the dental diagnostics summary 103 include tooth cracks, gum recession, tooth wear, occlusal contacts, crowding and/or spacing of teeth and/or other malocclusions, plaque, tooth stains, and caries. Additional, fewer and/or alternative dental conditions may also be analyzed and reported in the dental diagnostics summary 103. In at least one embodiment, multiple different types of analyses are performed to determine presence and/or severity of one or more of the dental conditions. One type of analysis that may be performed is a point-in-time analysis that identifies the presence and/or severity levels of one or more dental conditions at a particular point-in-time based on data generated at that point-in-time. For example, a single 3D model of a dental arch may be analyzed to determine whether, at a particular point-in-time, a patient's dental arch included any caries, gum recession, tooth wear, problem occlusion contacts, crowding, spacing or tooth gaps, plaque, tooth stains, and/or tooth cracks. Another type of analysis that may be performed is a time-based analysis that compares dental conditions at two or more points in time to determine changes in the dental conditions, progression of the dental conditions and/or rates of change of the dental conditions, as discussed with reference to FIG. 1B. For example, in at least one embodiment, a comparative analysis is performed to determine differences between 3D models of dental arches taken at different points in time. The differences may be measured to determine an amount of change, and the amount of change together with the times at which the intraoral scans that were used to generate the 3D models were taken may be used to determine a rate of change. This technique may be used, for example, to identify an amount of change and/or a rate of change for tooth wear, staining, plaque, crowding, spacing, gum recession, caries development, tooth cracks, and so on.

In at least one embodiment, one or more trained models are used to perform at least some of the one or more dental condition analyses. The trained models may include physics models and/or machine learning models, for example. In at least one embodiment, a single model may be used to perform multiple different analyses (e.g., to identify any combination of tooth cracks, gum recession, tooth wear, occlusal contacts, crowding and/or spacing of teeth and/or other malocclusions, plaque, tooth stains, and/or caries). Additionally, or alternatively, different models may be used to identify different dental conditions. For example, a first model may be used to identify tooth cracks, a second model may be used to identify tooth wear, a third model may be used to identify gum recession, a fourth model may be used to identify problem occlusal contacts, a fifth model may be used to identify crowding and/or spacing of teeth and/or other malocclusions, a sixth model may be used to identify plaque, a sixth model may be used to identify tooth stains, and/or a seventh model may be used to identify caries.

In at least one embodiment, intraoral data from one or more points in time are input into one or more trained machine learning models that have been trained to receive the intraoral data as an input and to output classifications of one or more types of dental conditions. In at least one embodiment, the trained machine learning model(s) is trained to identify areas of interest (AOIs) from the input intraoral data and to classify the AOIs based on dental conditions. The AOIs may be or include regions associated with particular dental conditions. The regions may include nearby or adjacent pixels or points that satisfy some criteria, for example. The intraoral data that is input into the one or more trained machine learning model may include three-dimensional (3D) data and/or two-dimensional (2D) data. The intraoral data may include, for example, one or more 3D models of a dental arch, one or more projections of one or more 3D models of a dental arch onto one or more planes (optionally comprising height maps), one or more x-rays of teeth, one or more CBCT scans, a panoramic x-ray, near-infrared and/or infrared imaging data, color image(s), ultraviolet imaging data, intraoral scans, and so on. If data from multiple imaging modalities are used (e.g., 3D scan data, color images, and NIRI imaging data), then the data may be registered and/or stitched together so that the data is in a common reference frame and objects in the data are correctly positioned and oriented relative to objects in other data. One or more feature vectors may be input into the trained model, where the feature vectors include multiple channels of information for each point or pixel of an image. The multiple channels of information may include color channel information from a color image, depth channel information from intraoral scan data, a 3D model or a projected 3D model, intensity channel information from an x-ray image, and so on.

The trained machine learning model(s) may output a probability map, where each point in the probability map corresponds to a point in the intraoral data (e.g., a pixel in an intraoral image or point on a 3D surface) and indicates probabilities that the point represents one or more dental classes. In at least one embodiment, a single model outputs probabilities associated with multiple different types of dental classes, which includes one or more dental condition classes. In an example, a trained machine learning model may output a probability map with probability values for a teeth dental class and a gums dental class. The probability map may further include probability values for tooth cracks, gum recession, tooth wear, occlusal contacts, crowding and/or spacing of teeth and/or other malocclusions, plaque, tooth stains, healthy area (e.g., healthy tooth and/or healthy gum) and/or caries. In the case of a single machine learning model that can identify each of tooth cracks, gum recession, tooth wear, occlusal contacts, crowding and/or spacing of teeth and/or other malocclusions, plaque, tooth stains, and caries, eleven valued labels may be generated for each pixel, one for each of teeth, gums, healthy area, tooth cracks, gum recession, tooth wear, occlusal contacts, crowding and/or spacing of teeth and/or other malocclusions, plaque, tooth stains, and caries. The corresponding predictions have a probability nature: for each pixel there are multiple numbers that may sum up to 1.0 and can be interpreted as probabilities of the pixel to correspond to these classes. In at least one embodiment, the first two values for teeth and gums sum up to 1.0 and the remaining values for healthy area, tooth cracks, gum recession, tooth wear, occlusal contacts, crowding and/or spacing of teeth and/or other malocclusions, plaque, tooth stains, and/or caries sum up to 1.0.

In some instances, multiple machine learning models are used, where each machine learning model identifies a subset of the possible dental conditions. For example, a first trained machine learning model may be trained to output a probability map with three values, one each for healthy teeth, gums, and caries. Alternatively, the first trained machine learning model may be trained to output a probability map with two values, one each for healthy teeth and caries. A second trained machine learning model may be trained to output a probability map with three values (one each for healthy teeth, gums and tooth cracks) or two values (one each for healthy teeth and tooth cracks). One or more additional trained machine learning models may each be trained to output probability maps associated with identifying specific types of dental conditions.

In case of an ML model trained to identify three classes, it is convenient to store such predictions of dental classes in an RGB format. For example, a first value for a first dental class may be stored as a red intensity value, a second value for a second dental class may be stored as a green intensity value, and a third value for a third dental class may be stored as a blue intensity value. This may make visualization of the probability map very easy. Usually, there is no need in high precision and chars can be used instead of floats—that is 256 possible values for every channel of the pixel. Further optimization can be done in order to reduce the size and improve performance (e.g., use 16 values quantization instead of 256 values).

The output of the one or more trained machine learning models may be used to update one or more versions of the 3D model of the patient's upper and/or lower dental arches. In at least one embodiment, a different layer is generated for each dental condition class. A layer may be turned on to graphically illustrate areas of interest on the upper and/or lower dental arch that has been identified or flagged as having a particular dental condition.

If the probability maps were generated for one or more input 2D images (e.g., such as height maps in which pixel intensity represents height or depth), the probability maps output by the ML model(s) may be projected onto the points in the virtual 3D model. Accordingly, each point in the virtual 3D model may include probability information from probability maps of one or multiple different intraoral images that map to that point. In at least one embodiment, the probability information from the probability map is projected onto the 3D model as a texture. The updated 3D model may then include, for one or more points, vertices or voxels of the 3D model (e.g., vertexes on a 3D mesh that represents the surface of the 3D model), multiple sets of probabilities, where different sets of probabilities associated with probability maps generated for different input images or other intraoral data may have different probability values.

Processing logic may modify the virtual 3D model by determining, for each point in the virtual 3D model, one or more dental class for that point. This may include using a voting function to determine a dental class for each point. For example, each set of probability values from an intraoral image may indicate a particular dental class. Processing logic may determine the number of votes for each dental class for a point, and may then classify the point as having a dental class that receives the most votes. In at least one embodiment, points may be associated with multiple classes of dental conditions.

In at least one embodiment, image processing and/or 3D data processing may be performed on 3D models of dental arches generated from intraoral scans and/or on the output of one or more trained models. Such image processing and/or 3D data processing may be performed using one or more algorithms, which may be generic to multiple types of dental conditions or may be specific to particular dental conditions. For example, a trained model may identify regions on a 3D model of a dental arch that include caries, and image processing may be performed to assess the size and/or severity of the identified caries. The image processing may include performing automated measurements such as size measurements, distance measurements, amount of change measurements, rate of change measurements, ratios, percentages, and so on. Accordingly, the image processing and/or 3D data processing may be performed to determine severity levels of dental conditions identified by the trained model(s). Alternatively, the trained models may be trained both to classify regions as caries and to identify a severity and/or size of the caries.

The one or more trained machine learning models that are used to identify, classify and/or determine a severity level for dental conditions may be neural networks such as deep neural networks or convolutional neural networks. Such machine learning models may be trained using supervised training in at least one embodiment.

Artificial neural networks (e.g., deep neural networks and convolutional neural networks) generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize that the image contains a face or define a bounding box around teeth in the image. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

In at least one embodiment, a U-net architecture is used. A U-net is a type of deep neural network that combines an encoder and decoder together, with appropriate concatenations between them, to capture both local and global features. The encoder is a series of convolutional layers that increase the number of channels while reducing the height and width when processing from inputs to outputs, while the decoder increases the height and width and reduces the number of channels. Layers from the encoder with the same image height and width may be concatenated with outputs from the decoder. Any or all of the convolutional layers from encoder and decoder may use traditional or depth-wise separable convolutions.

In at least one embodiment, the machine learning model is a recurrent neural network (RNN). An RNN is a type of neural network that includes a memory to enable the neural network to capture temporal dependencies. An RNN is able to learn input-output mappings that depend on both a current input and past inputs. The RNN will address past and future intraoral data (e.g., intraoral scans taken at different times) and make predictions based on information that spans multiple time periods and/or patient visits. RNNs may be trained using a training dataset to generate a fixed number of outputs. One type of RNN that may be used is a long short term memory (LSTM) neural network.

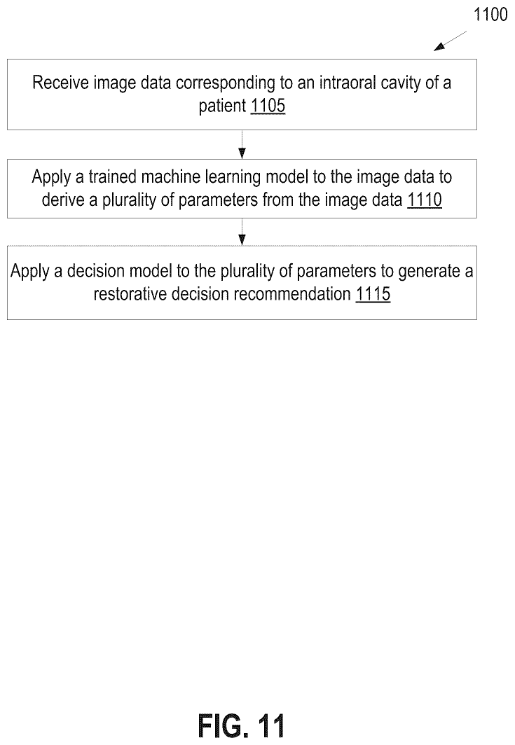

A common architecture for such tasks is LSTM (Long Short Term Memory). Unfortunately, LSTM is not well suited for images since it does not capture spatial information as well as convolutional networks do. For this purpose, one can utilize ConvLSTM—a variant of LSTM containing a convolution operation inside the LSTM cell. ConvLSTM is a variant of LSTM (Long Short-Term Memory) containing a convolution operation inside the LSTM cell. ConvLSTM replaces matrix multiplication with a convolution operation at each gate in the LSTM cell. By doing so, it captures underlying spatial features by convolution operations in multiple-dimensional data. The main difference between ConvLSTM and LSTM is the number of input dimensions. As LSTM input data is one-dimensional, it is not suitable for spatial sequence data such as video, satellite, radar image data set. ConvLSTM is designed for 3-D data as its input. In at least one embodiment, a CNN-LSTM machine learning model is used. A CNN-LSTM is an integration of a CNN (Convolutional layers) with an LSTM. First, the CNN part of the model processes the data and a one-dimensional result feeds an LSTM model. The network architecture for excess material removal may look as is shown in FIGS. 11A-B in at least one embodiment, which includes a ConvLSTM machine learning model.

In at least one embodiment, a class of machine learning model called a MobileNet is used. A MobileNet is an efficient machine learning model based on a streamlined architecture that uses depth-wise separable convolutions to build light weight deep neural networks. MobileNets may be convolutional neural networks (CNNs) that may perform convolutions in both the spatial and channel domains. A MobileNet may include a stack of separable convolution modules that are composed of depthwise convolution and pointwise convolution (conv 1×1). The separable convolution independently performs convolution in the spatial and channel domains. This factorization of convolution may significantly reduce computational cost from HWNK2M to HWNK2 (depthwise) plus HWNM (conv 1×1), HWN(K2+M) in total, where N denotes the number of input channels, K2 denotes the size of convolutional kernel, M denotes the number of output channels, and H×W denotes the spatial size of the output feature map. This may reduce a bottleneck of computational cost to conv 1×1.

In at least one embodiment, a generative adversarial network (GAN) is used. A GAN is a class of artificial intelligence system that uses two artificial neural networks contesting with each other in a zero-sum game framework. The GAN includes a first artificial neural network that generates candidates and a second artificial neural network that evaluates the generated candidates. The GAN learns to map from a latent space to a particular data distribution of interest (a data distribution of changes to input images that are indistinguishable from photographs to the human eye), while the discriminative network discriminates between instances from a training dataset and candidates produced by the generator. The generative network's training objective is to increase the error rate of the discriminative network (e.g., to fool the discriminator network by producing novel synthesized instances that appear to have come from the training dataset). The generative network and the discriminator network are co-trained, and the generative network learns to generate images that are increasingly more difficult for the discriminative network to distinguish from real images (from the training dataset) while the discriminative network at the same time learns to be better able to distinguish between synthesized images and images from the training dataset. The two networks of the GAN are trained once they reach equilibrium. The GAN may include a generator network that generates artificial intraoral images and a discriminator network that segments the artificial intraoral images. In at least one embodiment, the discriminator network may be a MobileNet.

In at least one embodiment, the machine learning model is a conditional generative adversarial (cGAN) network, such as pix2pix. These networks not only learn the mapping from input image to output image, but also learn a loss function to train this mapping. GANs are generative models that learn a mapping from random noise vector z to output image y, G: z→y. In contrast, conditional GANs learn a mapping from observed image x and random noise vector z, to y, G: {x, z}→y. The generator G is trained to produce outputs that cannot be distinguished from “real” images by an adversarially trained discriminator, D, which is trained to do as well as possible at detecting the generator's “fakes.” The generator may include a U-net or encoder-decoder architecture in at least one embodiment. The discriminator may include a MobileNet architecture in at least one embodiment. An example of a cGAN machine learning architecture that may be used is the pix2pix architecture described in Isola, Phillip, et al. “Image-to-image translation with conditional adversarial networks.” arXiv preprint (2017).

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

To train the one or more machine learning models, a training dataset (or multiple training datasets, one for each of the machine learning models to be trained) containing hundreds, thousands, tens of thousands, hundreds of thousands or more images should be used to form a training dataset. In at least one embodiment, up to millions of cases of patient dentition that include one or more labeled dental conditions such as cracked teeth, tooth wear, caries, gum recession, gum swelling, tooth stains, healthy teeth, healthy gums, and so on are used, where each case may include a final virtual 3D model of a dental arch (or other dental site such as a portion of a dental arch). The machine learning models may be trained to automatically classify and/or segment intraoral scans after an intraoral scanning session, and the segmentation/classification may be used to automatically determine presence and/or severity of dental conditions.

For each 3D model with labeled dental classes, a set of images (e.g., height maps) may be generated. Each image may be generated by projecting the 3D model (or a portion of the 3D model) onto a 2D surface or plane. Different images of a 3D model may be generated by projecting the 3D model onto different 2D surfaces or planes in at least one embodiment. For example, a first image of a 3D model may be generated by projecting the 3D model onto a 2D surface that is in a top down point of view, a second image may be generated by projecting the 3D model onto a 2D surface that is in a first side point of view (e.g., a buccal point of view), a third image may be generated by projecting the 3D model onto a 2D surface that is in a second side point of view (e.g., a lingual point of view), and so on. Each image may include a height map that includes a depth value associated with each pixel of the image. For each image, a probability map or mask may be generated based on the labeled dental classes in the 3D model and the 2D surface onto which the 3D model was projected. The probability map or mask may have a size that is equal to a pixel size of the generated image. Each point or pixel in the probability map or mask may include a probability value that indicates a probability that the point represents one or more dental classes. For example, there may be three dental classes, including a first dental class representing caries, a second dental class representing healthy teeth, and a third dental class representing gums. Points that have a first dental class may have a value of (1,0,0) (100% probability of first dental class and 0% probability of second and third dental classes), points that have a second dental class may have a value of (0,1,0), and points that have a third dental class may have a value of (0,0,1), for example.

A training dataset may be gathered, where each data item in the training dataset may include an image (e.g., an image comprising a height map) and an associated probability map. Additional data may also be included in the training data items. Accuracy of segmentation can be improved by means of additional classes, inputs and multiple views support. Multiple sources of information can be incorporated into model inputs and used jointly for prediction. Multiple dental classes can be predicted concurrently from a single model. Multiple problems can be solved simultaneously: teeth/gums segmentation, dental condition classification, etc. Accuracy is higher than traditional image and signal processing approaches.

Additional data may include a color image. For example, for each image (which may be a monochrome), there may also be a corresponding color image. Each data item may include depth information (e.g., a height map) as well as color information (e.g., from a color image). Two different types of color images may be available. One type of color image is a viewfinder image, and another type of color image is a scan texture. A scan texture may be a combination or blending of multiple different viewfinder images. Each intraoral scan may be associated with a corresponding viewfinder image generated at about the same time that the intraoral image was generated. If blended scans are used, then each scan texture may be based on a combination of viewfinder images that were associated with the raw scans used to produce a particular blended scan.

Another type of additional data may include an image generated under specific lighting conditions (e.g., an image generated under ultraviolet, near infrared or infrared lighting conditions). The additional data may be a 2D or 3D image, and may or may not include depth information (e.g., a height map).

The result of this training is a function that can predict dental classes directly from intraoral data (e.g., height maps of intraoral objects). In particular, the machine learning model(s) may be trained to generate a probability map, where each point in the probability map corresponds to a pixel of an input image and/or other input intraoral data and indicates one or more of a first probability that the pixel represents a first dental class, a second probability that the pixel represents a second dental class, a third probability that the pixel represents a third dental class, a fourth probability that the pixels represents a fourth dental class, a fifth probability that the pixel represents a fifth dental class, and so on.

From the dental diagnostics summary 103, a dentist may select any of the types of dental classes. For example, the dentist may select any one of tooth cracks 134, caries 120, gum recession 122, amalgam 124, occlusion 126, crowding/spacing 128, plaque 130 and/or tooth stains 132. As discussed, multiple different types of dental conditions may be displayed, and for each type of dental condition a severity level for that dental condition may be shown. In the illustrated example, caries 120, amalgam 124 and crowding/spacing 128 are shown to have issues found 155. Accordingly, as a result of performing a caries analysis, a tooth wear analysis and a crowding and/or spacing analysis, severity levels for tooth crowding and/or spacing 128, amalgam 124 and caries 120 exceeded respective severity level thresholds. In the illustrated example, tooth stains 132 and occlusion 126 (e.g., poor occlusal contacts) are shown to have potential issues found 150, and tooth cracks 134, gum recession 122 and plaque 130 are shown to have no issues found 145.

A dentist, after a quick glance at the dental diagnostics summary 103, may determine that a patient has carries, clinically significant tooth wear, and crowding/spacing and/or other malocclusions 128. Accordingly, the dentist may select the caries 120 view option, the amalgam 124 view option and/or the crowding/spacing 128 view option to quickly review the areas on the patient's dental arches at which caries, amalgam and/or crowding (and/or spacing) were detected. The dentist may determine not to review gum recession, tooth cracks or plaque for the patient due to these dental conditions being classified as having no issues found. The dentist may or may not review the tooth stains and occlusion information due to these dental conditions having been classified as having potential issues found. Each of the illustrated dental conditions may be shown with an icon, button, link, or selectable option that a user can select via a graphical user interface of the dental diagnostics hub. Clicking on or otherwise selecting a particular dental condition may enable one or more tools associated with that specific dental condition.

The tools available to assess a selected dental condition may depend on the dental condition selected. For example, different assessment tools may be available for tooth stains 132 than for caries 120. In general, one of the available tools associated with a selected dental condition includes a simulation of a prognosis of the dental condition. Via the simulation, a doctor may determine what the area of interest (or areas of interest) exhibiting the dental condition looked like in the past and what they are predicted to look like in the future.

In at least one embodiment, the dental diagnostics hub, and in particular the dental diagnostics summary 103, helps a doctor to quickly detect dental conditions and their respective severity levels, helps the doctor to make better judgments about treatment of dental conditions, and further helps the doctor in communicating with a patient that patient's dental conditions and possible treatments. This makes the process of identifying, diagnosing, and treating dental conditions easier and more efficient. The doctor may select any of the dental conditions to determine prognosis of that condition as it exists in the present and how it will likely progress into the future. Additionally, the dental diagnostics hub may provide treatment simulations of how the dental conditions will be affected or eliminated by one or more treatments.

In at least one embodiment, a doctor may customize the dental conditions and/or areas of interest by adding emphasis or notes to specific dental conditions and/or areas of interest. For example, a patient may complain of a particular tooth aching. The doctor may highlight that particular tooth on the 3D model of the dental arches. Dental conditions that are found that are associated with the particular highlighted or selected tooth may then be shown in the dental diagnostics summary. In a further example, a doctor may select a particular tooth (e.g., lower left molar), and the dental diagnostics summary may be updated by modifying the severity results to be specific for that selected tooth. For example, if for the selected tooth an issue was found for caries and a possible issue was found for tooth stains, then the dental diagnostics summary 103 would be updated to show no issues found for amalgam 124, occlusion 126, crowding/spacing 128, plaque 130, tooth cracks 134, and gum recession 122, to show a potential issue found for tooth stains 132 and to show an issue found for caries 120. This may help a doctor to quickly identify possible root causes for the pain that the patient complained of for the specific tooth that was selected. The doctor may then select a different tooth to get a summary of dental issues for that other tooth. Additionally, the doctor may select a dental arch, a quadrant of a dental arch, or a set of teeth, and the dental diagnostics summary 103 may be updated to show the dental conditions associated with the selected set of teeth, quadrant of a dental arch, and/or dental arch.

FIG. 1D illustrates a user interface for navigating diagnostics results provided to a mobile device 158 by a dental diagnostics hub, in accordance with at least one embodiment of the present disclosure. The dental diagnostics summary 103 generated by a dental diagnostics hub may be sent to a device of a patient, which may be a mobile device 158 or a traditionally stationary device. Examples of mobile devices include mobile phones, tablet computers, laptops, and so on. Examples of traditionally stationary devices include desktop computers, server computers, smart televisions, set top boxes, and so on. In at least one embodiment, a link to the dental diagnostics summary 103 may be sent to the device of the patient, and the patient may activate the link (e.g., click on the link) to access the dental diagnostics summary 103. In at least one embodiment, the underlying information that is summarized in the dental diagnostics summary 103 may also be accessible by the patient by selecting on one or more of the dental conditions in the dental diagnostics summary 103. This may show the patient which teeth exhibit specific dental conditions, for example. The patient's version of the dental diagnostics summary 103 may further include or be associated with a schedule appointment option or function 160. The patient may click on or otherwise select the schedule appointment option or function 160 to schedule an appointment. This may cause a mobile phone to call a dentist office for example, or may cause the patient's device to navigate to a calendar view showing available appointment times. The patient may click on or otherwise select an available appointment time to schedule an appointment with their dentist.

FIG. 1E illustrates a user interface of a dental diagnostics hub showing a dental diagnostics summary 161 after diagnostics have been run on a patient's dental arches, in accordance with at least one embodiment of the present disclosure. The dental diagnostics summary 161 presents dental information about a patient organized in a different manner than is shown in dental diagnostics summary 103. For dental diagnostics summary 103, summary information for many different types of dental conditions are shown together, without grouping the summary information based on dental categories. Dental diagnostics summary 161, on the other hand, groups dental conditions based on dental categories, and indicates specific types of dental conditions or problems within each of the dental categories. The dental condition information may also be arranged and presented in many other ways than the few examples shown herein.

In at least one embodiment, dental diagnostics summary 161 includes multiple high level dental categories or groups, including a restorative/prosthodontic category 162, a TMJ category 188, an orthodontic category 174, a periodontal category 164 and an endodontic category 182. All restorative and/or prosthodontic dental conditions may be displayed under restorative/prosthodontic category 162, all dental conditions associated with or caused by problems with TMJ may be displayed under TMJ category 188, all orthodontic dental conditions may be displayed under orthodontic category 174, all periodontal dental conditions may be displayed under periodontal category 164, and all endodontic dental conditions may be displayed under the endodontic category 182. Each of the high level dental categories may be coded (e.g., color coded) or otherwise include indicators to show whether or not dental conditions falling under those high level categories have been detected and/or severity levels of such dental conditions.

In at least one embodiment, one or more of the high level dental categories (e.g., restorative/prosthodontic category 162, TMJ category 188, orthodontic category 174, periodontal category 164 and endodontic category 182) include summary information for subcategories and/or particular dental conditions falling within the respective high level dental categories. In at least one embodiment, TMJ category 188 includes a cracks dental condition 190, an occlusion dental condition 192 and a tooth wear dental condition 194, which may correspond to tooth cracks 134, occlusion 126 and amalgam 124, respectively, of FIG. 1C. For each of the cracks dental condition 190, occlusion dental condition 192 and tooth wear dental condition 194, the dental diagnostics summary 161 may indicate whether teeth of the patient are affected by the respective dental condition, which teeth are affected by the respective dental condition and/or a severity of the respective dental condition. Each of dental conditions within the TMJ category 188 may be coded (e.g., color coded) or otherwise include indicators to show whether or not the respective dental conditions have been detected and/or severity levels of such dental conditions.

In at least one embodiment, orthodontic category 174 includes a crowding dental category 176, a spacing dental category 178 and a jaw discrepancies dental category 180. Crowding dental category 176 may correspond to crowding 128 of FIG. 1C. Spacing dental category 178 may provide information on gaps or spaces between teeth of a patient. Jaw discrepancies dental category 180 may include information on problems with a patient's jaw, such as how the jaw closes, overbite, underbite, overjet, and so on. For each of the crowding dental category 176, spacing dental category 178 and jaw discrepancies dental category 180, the dental diagnostics summary 161 may indicate whether teeth of the patient are affected by the respective dental condition, which teeth are affected by the respective dental condition and/or a severity of the respective dental condition. Each of dental conditions within the orthodontic category 174 may be coded (e.g., color coded) or otherwise include indicators to show whether or not the respective dental conditions have been detected and/or severity levels of such dental conditions. Selection of any of the spacing dental category 178, jaw discrepancies dental category 180 or crowding dental category 176 may launch a dental analysis tool illustrating the respective dental condition that was selected on the patient's dentition. From any of those dental analysis tools, an orthodontics tool may be launched.

In at least one embodiment, periodontal category 164 includes an inflammation dental category 166, a bone loss dental category 170 and a gum recession dental category 167. Gum recession dental category 167 may correspond to gum recession 122 of FIG. 1C. Inflammation dental category 166 may include information on gum swelling or inflammation for one or more teeth and/or a degree of swelling. Bone loss dental category 170 may include information on bone density loss for one or more regions of a patient's jaw. For each of the gum recession dental category 167, inflammation dental category 166 and bone loss dental category 170, the dental diagnostics summary 161 may indicate whether teeth of the patient are affected by the respective dental condition, which teeth are affected by the respective dental condition (e.g., tooth nos. 168, 169, 172) and/or a severity of the respective dental condition. Each of the dental conditions within the periodontal category 164 may be coded (e.g., color coded) or otherwise include indicators to show whether or not the respective dental conditions have been detected and/or severity levels of such dental conditions. Selection of any of the gum recession dental category 167, inflammation dental category 166 or bone loss dental category 170 may launch a dental analysis tool illustrating the respective dental condition that was selected on the patient's dentition.

In at least one embodiment, endodontic category 182 includes one or more types of endodontic problems. Endodontic problems may include problems relating to tooth roots and the soft tissues inside a tooth, such as dental pulp in a tooth. Endodontic category 182 may include endodontic conditions for one or more problem types 184, such as a first problem type for problems with dental pulp and a second problem type for problems with tooth roots. For each problem type 184, one or more affected tooth numbers 186 may be indicated. Each of dental conditions within the endodontic category 182 may be coded (e.g., color coded) or otherwise include indicators to show whether or not the respective dental conditions have been detected and/or severity levels of such dental conditions. Selection of any of the problem types 184 may launch a dental analysis tool illustrating the respective dental condition that was selected on the patient's dentition.

In at least one embodiment, restorative/prosthodontic category 162 includes one or more types of restorative and/or prosthodontic conditions. The term prosthodontic procedure refers, inter alia, to any procedure involving the oral cavity and directed to the design, manufacture or installation of a dental prosthesis at a dental site within the oral cavity, or a real or virtual model thereof, or directed to the design and preparation of the dental site to receive such a prosthesis. A prosthesis may include any restoration such as crowns, veneers, inlays, onlays, and bridges, for example, and any other artificial partial or complete denture. Prosthodontic dental conditions or issues may include a failing, failed or broken/cracked prosthesis, a worn prosthesis, a loose prosthesis, an ill-fitting prosthesis, and so on. Prosthodontic dental conditions may also include conditions that can be corrected by a prosthesis, such as a missing tooth, an edentulous dental arch, and so on. Restorative/prosthodontic category 162 may include conditions with existing prosthodontics, which may constitute a first problem type 163, and conditions that can be resolved using prosthodontics, which may constitute a second problem type 163. For each problem type 163, one or more affected tooth numbers 165 may be indicated. Each of the dental conditions within the restorative/prosthodontic category 162 may be coded (e.g., color coded) or otherwise include indicators to show whether or not the respective dental conditions have been detected and/or severity levels of such dental conditions. Selection of any of the problem types 163 may launch a dental analysis tool illustrating the respective dental condition that was selected on the patient's dentition.

In at least one embodiment, the dental diagnostics summary 161 includes the scan segment selector 102 including upper dental arch segment selection 105, lower dental arch segment selection 110 and/or bite segment selection 115. In at least one embodiment, the dental diagnostics summary 103 further includes views of the selected dental segments or modes (e.g., of the 3D model of the upper dental arch 140 and the 3D model of the lower dental arch 141 of the patient).

The dental diagnostics summary 161 provides a single view showing multiple different types of possible dental conditions at both a high level and at a lower level, and assessments as to the presence and/or severity of each of the types of dental conditions. In at least one embodiment, the various dental conditions are assigned one of three severity levels, including “no issues found,” “potential issues found,” and “issues found.” Each of the dental conditions and/or dental categories (e.g., high level categories that may include multiple underlying conditions) may be coded or labeled with the severity ranking determined for that type of dental condition. In at least one embodiment, the dental conditions and/or categories are color coded to graphically show severity levels. For example, those dental conditions and/or categories for which issues were found may be coded red, those dental conditions and/or categories for which potential issues were found may be coded yellow, and those dental conditions and/or categories for which no issues were found may be coded green. Many other coding schemes are also possible. In at least one embodiment, each of the dental conditions and/or categories is assigned a numeric severity level. For example, on a scale of 1 to 100, each dental condition and/or category may be assigned a severity level between 1 and 100 to indicate the severity level of that dental condition. Those dental conditions and/or categories with a severity level that is below a first threshold severity level may be identified as dental conditions for which no issues were found. Those dental conditions and/or categories for which the severity level is above the first threshold severity level but below a second threshold severity level may be identified as dental conditions/categories for which potential issues were found. Those dental conditions and/or categories for which the severity level is above the second severity level threshold may be identified as dental conditions/categories for which issues were found.

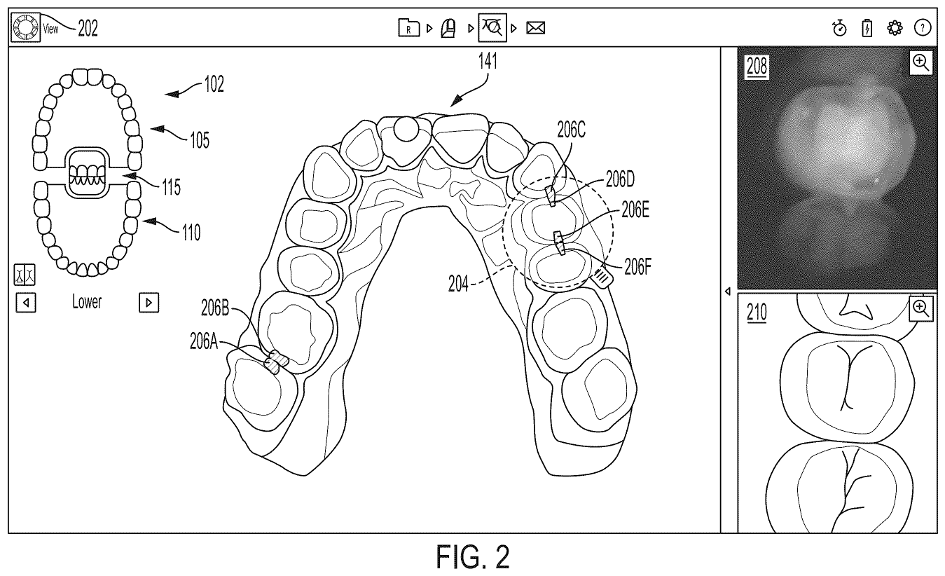

FIG. 2 illustrates a user interface for a caries analysis of a dental diagnostics hub, in accordance with at least one embodiment of the present disclosure. The user interface for caries analysis may be provided responsive to a doctor selecting caries 120 from the dental diagnostics summary 103. In at least one embodiment, the caries analysis user interface includes the scan segment selector 102 including upper dental arch segment selection 105, lower dental arch segment selection 110 and/or bite segment selection 115. In at least one embodiment, each tooth that has an area of interest associated with the selected dental condition is highlighted or otherwise emphasized or marked in the scan segment selector 102. Accordingly, a quick glance at the scan segment selector 102 may show a doctor where to look further to review the AOIs with the selected dental condition. In at least one embodiment, individual teeth may be selected in the scan segment selector 102 to view just those selected teeth. For example, a doctor may select one or a few teeth having AOIs to show a 3D model with just those teeth.

In at least one embodiment, the user interface for caries analysis further includes views of the selected dental segments or modes. In the illustrated example, a lower dental arch is selected, and the 3D model of the lower dental arch 141 of the patient is shown. An overlay of areas of interest (AOIs) that reflect detected caries is shown on the 3D model of the lower dental arch. For example, areas of interest 206A-F representing detected caries are shown on the 3D model of the lower dental arch 141. The upper dental arch segment may be selected to view AOIs representing caries in the upper dental arch. Additionally, both the upper and lower dental arch may be selected to show caries on both the upper and lower dental arches.

A doctor may change a view of the displayed 3D model or 3D models (e.g., of the 3D model of the lower dental arch 141) via the user interface so as to better view identified AOIs. Such changes to the view may include changing a zoom setting (e.g., by zooming in or out), rotating the 3D model(s), panning left, right, up, down, etc., and so on. A doctor may additionally use a focus tool to move a focus window 204 anywhere on the 3D model to focus in on a region of the 3D model of the dental arch(es). Additional information from one or more additional imaging modalities may be shown for a region that is within the focus window 204. For example, NIRI data for the region may be shown in a NIRI window 208, and color data for the region may be shown in a color window 210. For both the NIRI window 208 and the color window 210 the doctor may zoom in or out and/or change a view of the region.

The doctor may select a time-based simulation function to launch a time-based simulation for the selected dental condition (e.g., for caries). The time-based simulation may use information about AOIs as they existed at different points in time from the patient's record history (e.g., intraoral scans, NIRI images, color images, x-rays, etc. from different points in time) to project progression of the dental condition into the future and/or into the past. The time-based simulation may generate a video showing the start of the dental condition and progression of the dental condition over time to the present status of the dental condition and into the future. The time-based simulation may further include one or more treatment options, and may show what the areas of interest into the future after one or more selected treatments are performed.

The user interface for the caries analysis may indicate, for each of the detected caries 206A-F, a severity level of the caries. The severity level may be based on a size of the caries, on a location of the caries and/or on a distance between the caries and a patient's dentin and/or pulp.

In at least one embodiment, a secure share mode may be provided in which doctors can collaborate securely with other care providers and/or can communicate securely with patients (or parents of patients) via a remote connection.

A doctor may select a learn mode option (not shown) to bring up educational information on the difference between healthy teeth and teeth having caries, and the difference between different severity levels of caries. The patient's current dentition with currently detected caries may be shown, and further tooth decay may be projected. The educational information may show what happens when the tooth decay reaches the patient's dentin and/or pulp, indicating an amount of pain that the patient can expect at various stages of tooth decay. The educational information may be shown to a patient to show that patient the stages of tooth decay for their teeth and what will happen if they don't treat the tooth decay.

Once the doctor is done reviewing the caries information for the patient, the doctor may select a dental diagnostics summary view icon or navigation option 202 to navigate back to the dental diagnostic summary 103.

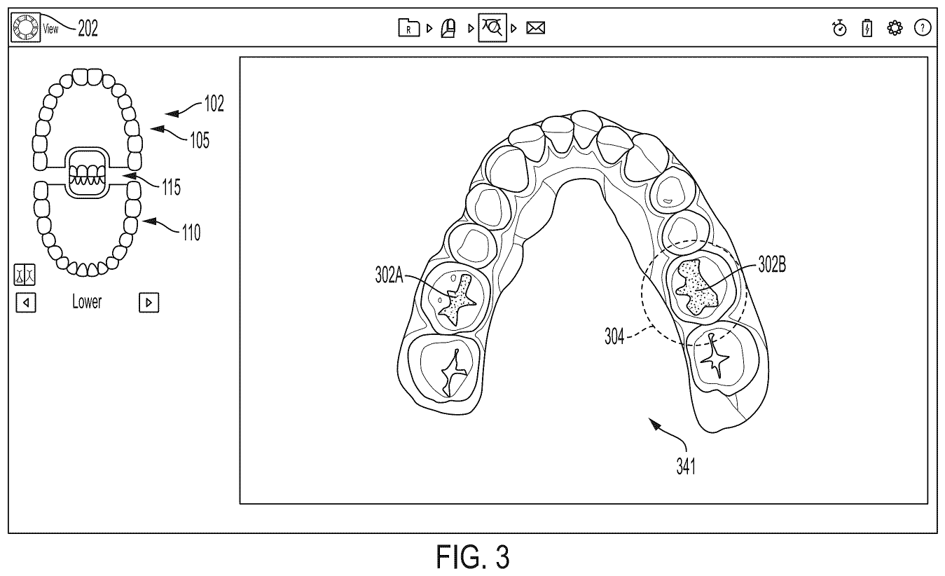

FIG. 3 illustrates a user interface for amalgam analysis of a dental diagnostics hub, in accordance with at least one embodiment of the present disclosure. The user interface for amalgam analysis may be provided responsive to a doctor selecting amalgam 124 from the dental diagnostics summary 103. In at least one embodiment, the amalgam analysis user interface includes the scan segment selector 102 including upper dental arch segment selection 105, lower dental arch segment selection 110 and/or bite segment selection 115. In at least one embodiment, each tooth that has an area of interest associated with the selected dental condition is highlighted or otherwise emphasized or marked in the scan segment selector 102. Accordingly, a quick glance at the scan segment selector 102 may show a doctor where to look further to review the AOIs with the selected dental condition. In at least one embodiment, individual teeth may be selected in the scan segment selector 102 to view just those selected teeth. For example, a doctor may select one or a few teeth having AOIs to show a 3D model with just those teeth.

In at least one embodiment, the user interface for amalgam analysis further includes views of the selected dental segments or modes. In the illustrated example, a lower dental arch is selected, and the 3D model of the lower dental arch 341 of the patient is shown. An overlay of areas of interest (AOIs) that reflect detected amalgam is shown on the 3D model of the lower dental arch. For example, areas of interest 302A-B representing detected regions where amalgam is present are shown on the 3D model of the lower dental arch 341. The upper dental arch segment may be selected to view AOIs representing amalgam in the upper dental arch. Additionally, both the upper and lower dental arch may be selected to show amalgam on both the upper and lower dental arches. A doctor may change a view of the displayed 3D model or 3D models (e.g., of the 3D model of a lower dental arch 341) via the user interface so as to better view identified AOIs. Such changes to the view may include changing a zoom setting (e.g., by zooming in or out), rotating the 3D model(s), panning left, right, up, down, etc., and so on. A doctor may additionally use a focus tool to move a focus window 304 anywhere on the 3D model to focus in on a region of the 3D model of the dental arch(es). Additional information from one or more additional imaging modalities may be shown for a region that is within the focus window 304. Once the doctor is done reviewing the tooth wear information for the patient, the doctor may select the dental diagnostics summary view icon or navigation option 202 to navigate back to the dental diagnostic summary 103.

Other types of user interfaces are also contemplated such, for example, for tooth wear analysis, occlusal contact analysis, malocclusion analysis, tooth stain analysis, and post-bleaching tooth. Such user interfaces may be similar to those described in U.S. Patent Publication No. 2022/0202295, the disclosure of which is hereby incorporated by reference herein in its entirety. Similar views for gum swelling, plaque, tooth cracks and/or gum recession may be shown to a dentist as are shown with regards to caries and tooth wear. Additionally, similar dental condition analysis tools may be provided for gum swelling, plaque, tooth cracks and/or gum recession as are provided for caries and/or tooth wear. A gum swelling analysis tool, for example, may project an amount of gum swelling into the future, and show inflammation of the gums, gum bleeding, and so on. Similarly, a gum recession analysis tool may project an amount of gum recession into the future, showing exposed portions of tooth roots, and so on.

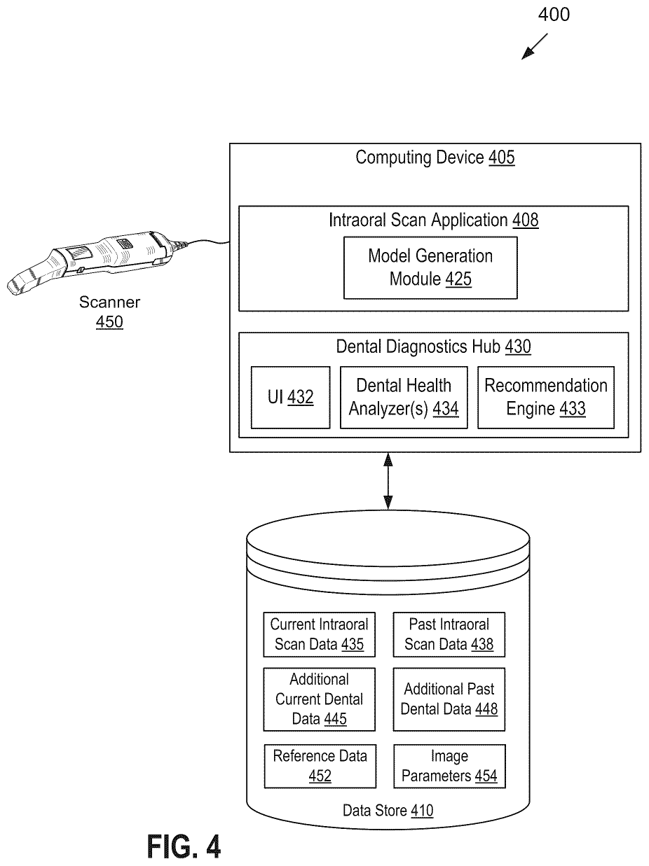

FIG. 4 illustrates one embodiment of a system 400 for performing intraoral scanning, generating a virtual three dimensional model of a dental site and/or performing dental diagnostics. In at least one embodiment, system 400 carries out one or more operations of below described with reference to FIGS. 1A-3 and 5-11. System 400 includes a computing device 405 that may be coupled to a scanner 450 and/or a data store 410.

Computing device 405 may include a processing device, memory, secondary storage, one or more input devices (e.g., such as a keyboard, mouse, tablet, and so on), one or more output devices (e.g., a display, a printer, etc.), and/or other hardware components. Computing device 405 may be connected to a data store 410 either directly or via a network. The network may be a local area network (LAN), a public wide area network (WAN) (e.g., the Internet), a private WAN (e.g., an intranet), or a combination thereof. The computing device 405 may be integrated into the scanner 450 in some embodiments to improve performance and mobility.

Data store 410 may be an internal data store, or an external data store that is connected to computing device 405 directly or via a network. Examples of network data stores include a storage area network (SAN), a network attached storage (NAS), and a storage service provided by a cloud computing service provider. Data store 410 may include a file system, a database, or other data storage arrangement.

In at least one embodiment, a scanner 450 for obtaining three-dimensional (3D) data of a dental site in a patient's oral cavity is operatively connected to the computing device 405. Scanner 450 may include a probe (e.g., a hand held probe) for optically capturing three dimensional structures (e.g., by confocal focusing of an array of light beams). One example of such a scanner 450 is the iTero® intraoral digital scanner manufactured by Align Technology, Inc. Other examples of intraoral scanners include the 8M™ True Definition Scanner and the Apollo DI intraoral scanner and CEREC AC intraoral scanner manufactured by Sirona®.

The scanner 450 may be used to perform an intraoral scan of a patient's oral cavity. An intraoral scan application 408 running on computing device 405 may communicate with the scanner 450 to effectuate the intraoral scanning. A result of the intraoral scanning may be a sequence of intraoral images or scans that have been generated. Each intraoral scan may include x, y and z position information for one or more points on a surface of a scanned object. In at least one embodiment, each intraoral scan includes a height map of a surface of a scanned object. An operator may start a scanning operation with the scanner 450 at a first position in the oral cavity, move the scanner 450 within the oral cavity to a second position while the scanning is being performed, and then stop recording of intraoral scans. In at least one embodiment, recording may start automatically as the scanner identifies either teeth. The scanner 450 may transmit the intraoral scans to the computing device 405. Computing device 405 may store the current intraoral scan data 435 from a current scanning session in data store 410. Data store 410 may additionally include past intraoral scan data 438, additional current dental data 445 generated during a current patient visit (e.g., x-ray images, CBCT scan data, panoramic x-ray images, ultrasound data, color photos, and so on), additional past dental data generated during one or more prior patient visits (e.g., x-ray images, CBCT scan data, panoramic x-ray images, ultrasound data, color photos, and so on), and/or reference data 452. Alternatively, scanner 450 may be connected to another system that stores data in data store 410. In such an embodiment, scanner 450 may not be connected to computing device 405.

According to an example, a user (e.g., a practitioner) may subject a patient to intraoral scanning. In doing so, the user may apply scanner 450 to one or more patient intraoral locations. The scanning may be divided into one or more segments (e.g., upper dental arch, lower dental arch, and bite). Via such scanner application, the scanner 450 may provide the current intraoral scan data 435 to computing device 405. The current and/or past intraoral scan data 435, 438 may include 3D surface data (e.g., in the form of 3D images or images with height information), 2D or 3D color image data, NIRI image data, ultraviolet image data, and so on. Such scan data may be provided from the scanner to the computing device 405 in the form of one or more points (e.g., one or more pixels and/or groups of pixels). For instance, the scanner 450 may provide a 3D image as one or more point clouds.

In at least one embodiment, intraoral scan application 408 includes a model generation module 425. When a scan session is complete (e.g., all images for a dental site have been captured), model generation module 425 may generate a virtual 3D model of the scanned dental site. To generate the virtual model, model generation module 425 may register and “stitch” together the intraoral scans generated from the intraoral scanning session. In at least one embodiment, performing registration includes capturing 3D data of various points of a surface in multiple scans (views from a camera), and registering the scans by computing transformations between the images, as discussed herein above.

In at least one embodiment, computing device 405 includes a dental diagnostics hub 430, which may include a UI 432, one or more dental health analyzers 434, and a recommendation engine 433. The UI 432 may be a graphical user interface and may include icons, buttons, graphics, menus, windows and so on for controlling and navigating the dental diagnostics hub 439.

Each of the dental health analyzers 434 may be responsible for performing an analysis associated with a different type of dental condition. For example, dental health analyzers 434 may include separate dental health analyzers 434 for tooth cracks, gum recession, tooth wear, occlusal contacts, crowding of teeth and/or other malocclusions, plaque, tooth stains, and/or caries. In at least one embodiment, a single dental health analyzer 434 performs each of the different type of dental health analyses associated with each of the types of dental conditions discussed herein. In at least one embodiment, there are multiple dental health analyzers, some of which perform dental health analysis for multiple different dental conditions. As discussed above, current intraoral scan data 435, past intraoral scan data 438, additional current dental data 445, additional past dental data 448 and/or reference data 452 may be used to perform one or more dental analysis. For example, the data regarding an at-hand patient may include X-rays, 2D intraoral images, 3D intraoral images, 2D models, and/or virtual 3D models corresponding to the patient visit during which the scanning occurs. The data regarding the at-hand patient may additionally include past X-rays, 2D intraoral images, 3D intraoral images, 2D models, and/or virtual 3D models of the patient (e.g., corresponding to past visits of the patient and/or to dental records of the patient).

Reference data 452 may include pooled patient data, which may include X-rays, 2D intraoral images, 3D intraoral images, 2D models, and/or virtual 3D models regarding a multitude of patients. Such a multitude of patients may or may not include the at-hand patient. The pooled patient data may be anonymized and/or employed in compliance with regional medical record privacy regulations (e.g., the Health Insurance Portability and Accountability Act (HIPAA)). The pooled patient data may include data corresponding to scanning of the sort discussed herein and/or other data. Reference data may additionally or alternatively include pedagogical patient data, which may include X-rays, 2D intraoral images, 3D intraoral images, 2D models, virtual 3D models, and/or medical illustrations (e.g., medical illustration drawings and/or other images) employed in educational contexts.

One or more dental health analyzers 434 may perform one or more types of dental condition analyses using intraoral data (e.g., current intraoral scan data 435, past intraoral scan data 438, additional current dental data 445, additional past dental data 448 and/or reference data 452), as discussed herein above. As a result, dental diagnostics hub 430 may determine multiple different dental conditions and severity levels of each of those types of identified dental conditions. In at least one embodiment, dental health analyzers 434 additionally use information of multiple different types of identified dental conditions and/or associated severity levels to determine correlations and/or cause and effect relationships between two or more of the identified dental conditions. Multiple dental conditions may be caused by the same underlying root cause. Additionally, some dental conditions may serve as an underlying root cause for other dental conditions. Treatment of the underlying root cause dental conditions may mitigate or halt further development of other dental conditions. For example, malocclusion (e.g., tooth crowding and/or tooth spacing or gaps), tooth wear and caries may all be identified for the same tooth or set of teeth. Dental diagnostics hub 430 may analyze these identified dental conditions that have a common, overlapping or adjacent area of interest, and determine a correlation or causal link between one or more of the dental conditions. In example, dental diagnostics hub 430 may determine that the caries and tooth wear for a particular group of teeth is caused by tooth crowding for that group of teeth. By performing orthodontic treatment for that group of teeth, the malocclusion may be corrected, which may prevent or reduce further caries progression and/or tooth wear for that group of teeth. In another example, plaque, tooth staining, and gum recession may be identified for a region of a dental arch. The tooth staining and gum recession may be symptoms of excessive plaque. The dental diagnostics hub 430 may determine that the plaque is an underlying cause for the tooth staining and/or gum recession.

In at least one embodiment, currently identified dental conditions may be used by the dental diagnostics hub 430 to predict future dental conditions that are not presently indicated. For example, a heavy occlusal contact may be assessed to predict tooth wear and/or a tooth crack in an area associated with the heavy occlusal contact. Such analysis may be performed by inputting intraoral data (e.g., current intraoral data and/or past intraoral data) and/or the dental conditions identified from the intraoral data into a trained machine learning model that has been trained to predict future dental conditions based on current dental conditions and/or current dentition (e.g., current 3D surfaces of dental arches). The machine learning model may be any of the types of machine learning models discussed elsewhere herein. The machine learning model may output a probability map indicating predicted locations of dental conditions and/or types of dental conditions. Alternatively, the machine learning model may output a prediction of one or more future dental conditions without identifying where those dental conditions are predicted to be located.

A recommendation engine 433 may be used to provide restorative decision recommendations based on image parameters 454 that are derived from current intraoral scan data 435, past intraoral scan data 438, and other information or image data. In at least one embodiment, the recommendation engine 433 utilizes a decision model that can generate, as outputs, restorative decision recommendations based on several parameters that are derived (e.g., automatically measured or approximated) from image data obtained through one or more of the following modalities: intraoral scan with, for example, NIRI, UV, or white light imaging (standard colors), radiographs (e.g., panoramic, bitewing, or periapical), or CBCT.

In at least one embodiment, the parameters useful for generating the restorative decision recommendations can be computed, for example, by applying a trained machine learning model to the images of the various modalities. Exemplary parameters include, but are not limited to, those described below in Table 1. As shown, different imaging modalities may be used to obtain the same types of parameters. For example, inter-cuspal width could potentially be estimated/computed from a 3D intraoral scan or, alternatively, from CBCT images or photographs/projections. Thus, in at least one embodiment, the restorative decision generated by the recommendation engine 433 may be agnostic as to the modality from which the parameters are derived, allowing for flexibility in selecting or combining imaging modalities to obtain the parameters useful for generating the recommendations.

TABLE 1
Exemplary parameters and their associated measurement techniques

| | EXEMPLARY | |
|---|---|---|
| | MEASUREMENT | ALTERNATE/ADDITIONAL |
| PARAMETER | TECHNIQUE | MEASUREMENT TECHNIQUE(S) |
| Inter-cuspal width: width | 3D intraoral scan can | CBCT |
| of the tooth between the | provide the inter-cuspal | or |
| buccal and lingual cusps. | width in mm | Photos or projections from an intraoral |
| Current restorative | | scanner can be used to estimate the |
| objects/material (e.g., | | width in pixels. Then the restorative |
| amalgam) can be measured | | inter-cuspal width can be measured in |
| as a percentage of the | | the same units and still expressed as a |
| intercuspal width. | | percentage |
| Restorative Volume | CBCT to identify the | Segment (e.g, using neural networks, |
| Proportion (RVP): the | tooth crown and the | computer vision, or deep learning) |
| proportion of a tooth crown | restorative objects in | occlusal restorative material from the |
| that is currently restorative | 3D. RVP is then the | intraoral scan (white light for amalgam |
| material | volume of restorative/ | and gold filling, or potentially UV light |
| | volume of the tooth | for composite). |
| | crown | Segment (e.g., using neural networks, |
| | | computer vision, or deep learning) |
| | | restorative material from an X-ray (e.g., |
| | | bite-wing, panoramic, or periapical). |
| | | Find the proportion of occlusal and |
| | | buccal-lingual surfaces that are |
| | | restorative to find the RVP as the |
| | | (occlusal surface proportion) * (x-ray |
| | | surface proportion) |
| | | or |
| | | Segment (e.g, using neural networks, |
| | | computer vision, or deep learning), |
| | | restorative material from the intraoral |
| | | scan (white light for amalgam and gold |
| | | filling, potentially UV light for |
| | | composite) for each visible tooth surface |
| | | (occlusal, buccal, or lingual). Estimate |
| | | the RVP using a regression from the |
| | | three surface proportions to the true |
| | | RVP |
| Decay Volume Proportion | Identify caries on the | If multiple modalities do not show the |
| (DVP): the proportion of | tooth crown (using a | caries, then approximate the DVP using |
| the tooth crown that is | similar segmentation | a single modality on a given surface |
| currently decayed | approach to finding | or |
| | RVP) but with NIRI | CBCT to identify decay |
| | imaging from the | |
| | intraoral scan and/or X- | |
| | ray images (bitewing, | |
| | panoramic, or | |
| | periapical) | |
| Restorative Surface | Segment (e.g., using | Segmentation using CBCT |
| Proportion (RSP): the | neural networks, | |
| proportion of a given tooth | computer vision, or deep | |
| crown surface (occlusal, | learning) occlusal | |
| buccal, or lingual) that is | restorative material from | |
| restoration | the intraoral scan (white | |
| | light for amalgam and | |
| | gold filling, potentially | |
| | UV light for composite). | |
| | The total surface area of | |
| | the restorative divided | |
| | by the total surface area | |
| | of the crown surface is | |
| | the RSP | |
| Classification of | CBCT segmentation | Visible light or geometric information in |
| Fractures: if a tooth has a | (using 3D neural | an intraoral scan can be analyzed by |
| fracture, designate as | networks) can provide | neural networks, computer vision, or |
| whether it is across the | information regarding a | geometric analysis to classify fractures |
| tooth (e.g., buccal-lingual or | fracture's location and | or |
| mesial-distal), only across a | depth | X-rays can be used to identify fractures |
| cusp or tip of the tooth, or if | | in crowns and teeth with root canals |
| it is a subsurface | | |

In at least one embodiment, the recommendation engine 433 may utilize one or more of a decision tree, a neural network, or other classifier to recommend a restorative type. In at least one embodiment, a decision tree may be utilized. An exemplary decision tree for illustrative purposes is now described:
If (inter-cuspal width of existing restorative>X %) AND ((% RVP+% DVP)>Y %) then generate warning about need for crown/onlay
Else, if (inter-cuspal width of existing restorative>X %) OR ((% RVP+% DVP)>Y %) then generate caution about need for crown/onlay
Else, if (inter-cuspal width of existing restorative<X %) AND ((% RVP+% DVP)>Y %) then
   1. If (% DVP>0) then direct restoration (filling) is recommended
   2. Otherwise, no restoration is recommended

In at least one embodiment, X % and Y % can be doctor/clinician-tunable parameters. In at least one embodiment, the parameters X % and Y % are determined based on a trained machine learning model that uses as inputs past restorative decisions and the parameters (e.g., % RVP, % DVP) derived from intraoral scan data. It is contemplated that other decision trees may be designed to recommend, for example, inlay versus onlay versus crown as restorative decisions based on the measured parameters of the tooth.

In at least one embodiment, restorative volume proportion (RVP) and restorative surface proportion (RSP) are parameters that may be derived from image data, for example, using trained machine learning models. For example, starting with a 2D rendering of a 3D model (e.g., generated by the model generation module 425 from data obtained from the scanner 450), a machine learning model may be trained to segment the teeth and detect/identify restorative objects/material present on the surface of each tooth. The RVP and RSP can then be determined from the areas of overlap, as discussed below, and can be utilized in a decision tree model together with indications of caries or other structural damage detected on the teeth. While amalgam is discussed below for illustrative purposes, other types of restorative objects/materials are contemplated.

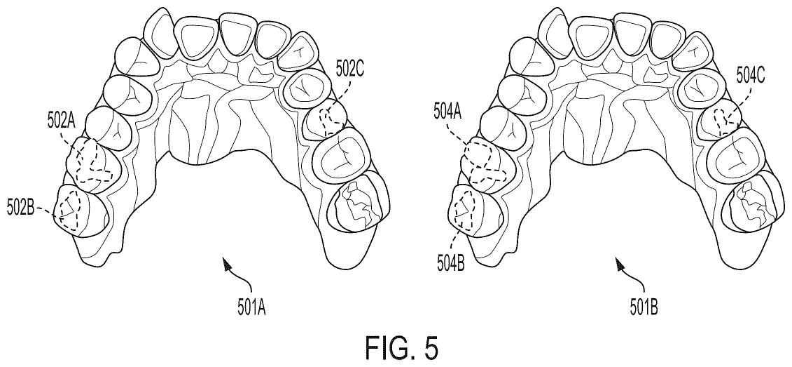

FIGS. 5-7 illustrate detection of amalgam present within 3D dentition models representative of a patient's intraoral cavity. In at least one embodiment, rendered images of 3D models (e.g., buccal, lingual, occlusal views) are first generated. The restorative material present in each view can then be labeled and used to train a machine learning model for segmentation of restorative material. In at least one embodiment, the model is applied to 2D renderings, and then projected onto the corresponding 3D mesh from which the 2D renderings are generated to perform the segmentation. FIG. 5 illustrates a 3D model of a lower dental arch comparing a prediction 501A to a labeled model 501B to show the quality of the segmentation between the segmented amalgam 502A-D and the labeled amalgam 504A-D. Larger amalgam regions are likely to be segmented more accurately than smaller regions and are less likely to return false positives on existing crowns, for example.

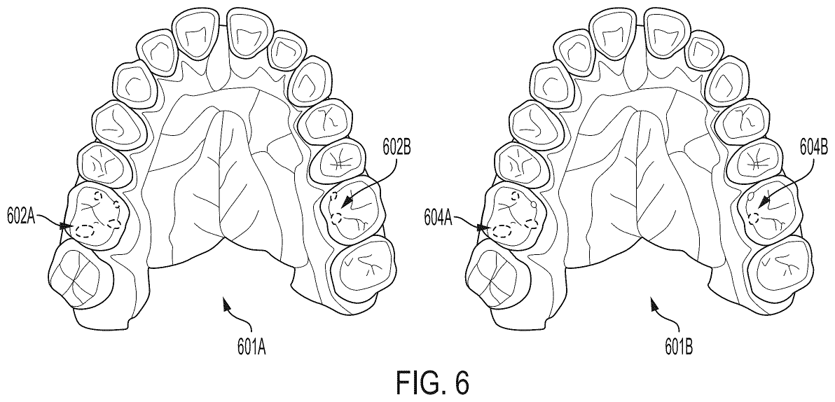

FIG. 6 illustrates classification of tooth decay regions comparing a prediction 601A to a labeled model 601B, showing a comparison of the segmented tooth decay regions 602A-B to labeled tooth decay regions 604A-B. Training the machine learning model to identify tooth decay regions can advantageously prevent misclassification of tooth decay regions as amalgam. Even in the situation where such misclassification occurs, the overall area may be small enough so as to have a minimal impact on restorative decisions.

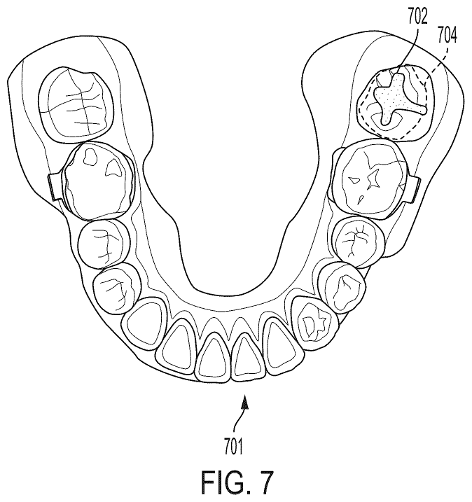

In at least one embodiment, RSP may be computed as a ratio between an area represented by restorative material over an area of an occlusal surface of the tooth. For example, FIG. 7 illustrates the segmentation of identified restorative material and an occlusal surface of a tooth. A 2D rendering of lower dental arch 701 is segmented to identify an restorative material region 702 (e.g., amalgam) and an occlusal surface region 704. The % RSP may be computed, for example, as the ration between the number of pixels within the region 702 divided by the number of pixels contained within the region 704. In at least one embodiment, one or more models may be used to determine if the patient is a candidate for restorative work, such as a crown. For example, in a heuristic model, if the % RSP is greater than a threshold amount, a recommendation for a crown may be generated.

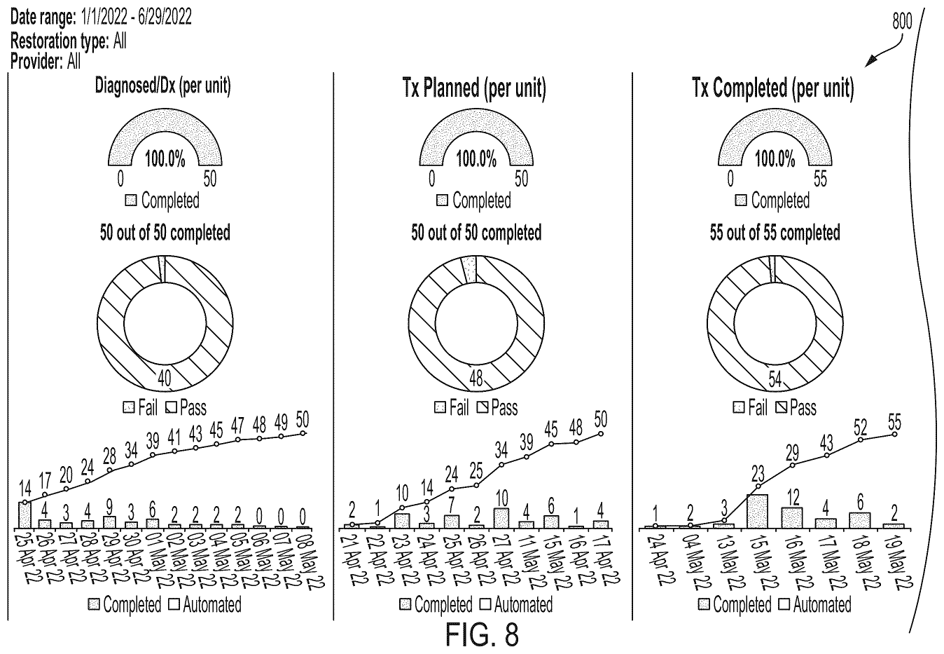

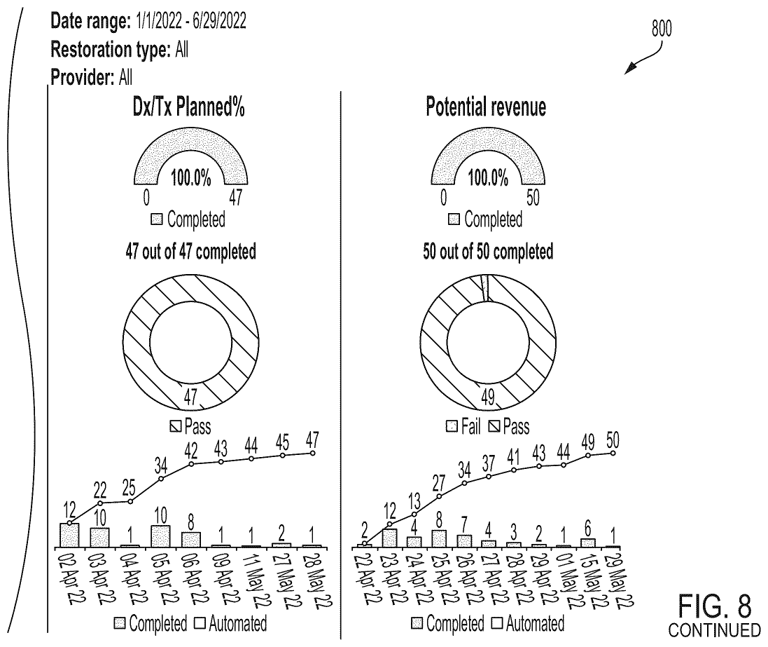

FIG. 8 illustrates a user interface for a key performance indicator (KPI) dashboard, in accordance with at least one embodiment of the present disclosure. The KPI dashboard may implemented as part of any of the dental diagnostics hub embodiments described herein, or may be utilized as part of a dental practice management system. In at least one embodiment, the information presented and visualized in the KPI dashboard may performance metrics at the level of individual doctors, classes of doctors (specialties), clinic, or region to track the outcomes of their respective patients. The outcomes include restorative types (such as direct restoration, crowns, inlays, and onlays), restoration counts (including the number of restorations diagnosed, treatment planned, and treatment completed/billed), and information related to results versus goals (e.g., diagnosed/treatment planned percent versus percent goal, diagnosed/treatment completed percent versus percent goal, and treatment planned/treatment completed percent versus percent goal). In addition, for each record, an associated restorative decision recommendation may be provided as well as an indication of whether the doctor followed the recommendation. The KPI dashboard can then be used as a more objective measure of treatment outcomes, and to evaluate semi-quantitatively whether a particular doctor's or clinic's treatment plans are consistent with the philosophy or standards set by the dental support organizations (DSOs). In at least one embodiment, the KPI dashboard can be extended to include additional services beyond restorative decision outcomes, such as revenue projection.

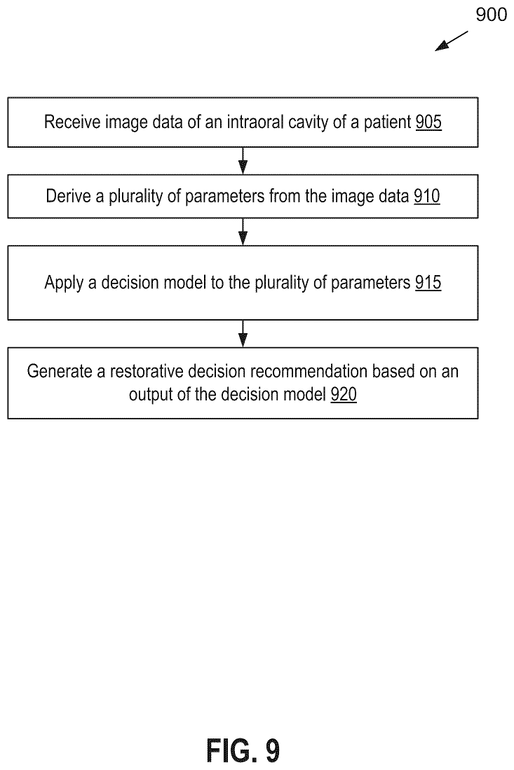

FIGS. 9-11 illustrate flow diagrams of methods performed by a dental diagnostics hub, in accordance with at least one embodiment of the present disclosure. These methods may be performed by processing logic that comprises hardware (e.g., circuitry, dedicated logic, programmable logic, microcode, etc.), software (such as instructions run on a processing device), or a combination thereof. In at least one embodiment, processing logic corresponds to computing device 405 of FIG. 4 (e.g., to a computing device 405 executing an intraoral scan application 408 and/or a dental diagnostics hub 430).

FIG. 9 illustrates a flow diagram for a method 900 of generating a restorative decision recommendation, in accordance with at least one embodiment of the present disclosure. At block 905, processing logic receives current, most recent, or previous image data of a patient's intraoral cavity. In at least one embodiment, the image data corresponds to one or more imaging modalities comprising, for example, an imaging modality selected from an intraoral scan, a radiograph, or a cone-beam computed tomography (CBCT) scan. In at least one embodiment, the one or more imaging modalities comprises the intraoral scan, and wherein the image data comprises one or more three-dimensional (3D) point clouds and at least one of two-dimensional (2D) near infrared (NIR) images, 2D ultraviolet images, or 2D color images. In at least one embodiment, the one or more imaging modalities comprise a radiograph and one or more of a panoramic radiograph, a bitewing radiograph, or a periapical radiograph. In at least one embodiment, the image data corresponds to two, three, or more imaging modalities.

In at least one embodiment, the image data are received as a result of a doctor or dental practitioner scanning the patient's intraoral cavity. The scan data may include 3D scan data (e.g., color or monochrome 3D scan data, which may be received in the form of point clouds, height maps, images, or other data types) and/or one or more 3D models of the patient's dental arches generated based on the scan data. The scan data may further include NIRI images and/or color images. The 3D scan data, NIRI images and/or color images 912 may all be generated by an intraoral scanner. In at least one embodiment, a doctor or dental practitioner may generate additional patient data. In at least one embodiment, processing logic receives the additional patient data. The additional patient data may include x-ray images (e.g., bitewing x-ray images and/or panoramic x-ray images) and/or dental information (e.g., such as observations of the dental practitioner, biopsy results, CBCT scan data, ultrasound data, etc.). In at least one embodiment, processing logic may import patient records for the patient being scanned. The imported patient records may include historical patient data such as historical NIRI images, color images, 3D scan data or 3D models generated from such 3D scan data, x-ray images and/or other information.

In at least one embodiment, processing logic may generate a 3D model of a current or more recent version of one or more dental arch of the patient using the current or most recent scan data and/or additional current or most recent dental data (e.g., using the model generation module 425). Alternatively, the 3D model may already have been generated, and the current or most recent scan data received may include the 3D model of the dental arch(es).

At block 910, processing logic derives a plurality of parameters from the image data. For example, a trained machine learning model may be utilized to segment regions of the image data, and are then used to compute various physical parameters. In at least one embodiment, the parameters are each selected from a geometric parameter, a volume/area parameter, or a fracture classification parameter. For example, the geometric parameter may comprise an inter-cuspal width. As a further example, the volume/area parameter may comprise one or more of a restorative volume proportion, a decay volume proportion, or a restorative surface proportion. As a further example, the fracture classification parameter may comprise information descriptive of a tooth fracture location and a tooth fracture depth.

At block 915, processing logic applies a decision model to the plurality of parameters. In at least one embodiment, the decision model comprises one or more of a decision tree or a neural network. In an exemplary embodiment, a decision tree may be utilized that includes tunable parameters (e.g., heuristic parameters) that may be used to determine if various threshold conditions are met. The threshold conditions may be used to determine which types of recommendations are generated.

At block 920, processing logic generates a restorative decision recommendation based on an output of the decision model. In at least one embodiment, the restorative decision recommendation comprises a direct restoration recommendation (e.g., a filling) or an indirect restoration recommendation (e.g., an inlay, onlay, crown, bridge, or veneer). In at least one embodiment, the restorative decision recommendation is presented for display in a GUI (e.g., the UI 432 of the dental diagnostics hub 430). The information presented in the user interface may include qualitative results and/or quantitative results of the various analyses. In at least one embodiment, a dental diagnostics summary is shown that includes high level results, but that does not include low level details or detailed information underlying the high level results. All of the results of the analyses may be presented together in a unified view that improves clinical efficiency and provides for improved communication between the doctor and patient about the patient's oral health and how best to treat dental conditions.

In at least one embodiment, the restorative decision recommendation comprises an indication of a dental condition and a severity level for the dental condition. For example, dental condition(s) may include, but is not limited to, caries, gum recession, tooth wear, malocclusion, tooth crowding, tooth spacing, plaque, tooth stains, tooth cracks, cervical defects (abfractions), or chipped or broken teeth. In at least one embodiment, processing logic determines a suggested treatment. Each of the types of dental conditions may be associated with one or more standard treatments that are performed in dentistry and/or orthodontics to treat that type of dental condition. Based on the locations of identified AOIs, the dental conditions for the identified AOIs, the number of AOIs having dental conditions and/or the severity levels of the dental conditions, a treatment plan may be suggested. A doctor may review the treatment plan and/or adjust the treatment plan based on their practice and/or preferences. In at least one embodiment, the doctor may customize the dental diagnostics hub to give preference to some types of treatment options over other types of treatment options based on the doctor's preferences. Treatments may be determined for each of the identified dental conditions that are determined to have clinical significance.

In at least one embodiment, processing logic generates diagnostics results based on an outcome of the dental condition analyses performed and recommendations generated previously. Processing logic may generate caries results, discoloration results, malocclusion results, tooth wear results, gum recession results, plaque results, gum swelling results, tooth crowding and/or spacing results and/or tooth crack results. The diagnostics results may include detected AOIs associated with each of the types of dental conditions, and severity levels of the dental conditions for the AOIs. The diagnostics results may include qualitative measurements, such as size of an AOI, an amount of recession for a gum region, an amount of wear for a tooth region, and amount of change (e.g., for a caries, tooth wear, gum swelling, gum recession, tooth discoloration, etc.), a rate of change (e.g., for a caries, tooth wear, gum swelling, gum recession, tooth discoloration, etc.), and so on. The diagnostics results may further include qualitative results, such as indications as to whether a dental condition at an AOI has improved, has stayed the same, or has worsened, indications as to the rapidity with which the dental condition has improved or worsened, an acceleration in the improvement or worsening of the dental condition, and so on. An expected rate of change may have been determined (e.g., automatically or with doctor input), and the measured rate of change for a dental condition at an AOI may be compared to the expected rate of change. Differences between the expected rate of change and the measured rate of change may be recorded and included in the diagnostics results. Each of the diagnostics results may be automatically assigned a code on dental procedures and nomenclature (CDT) code or other procedural code for health and adjunctive services provided in dentistry. Each of the diagnostics results may automatically be assigned an appropriate insurance code and related financial information.

In at least one embodiment, processing logic stores, in a record associated with a dentist to whom the restorative decision recommendation was provided, the restorative decision recommendation and an actual restorative decision made by the dentist in a key performance indicator (KPI) database.

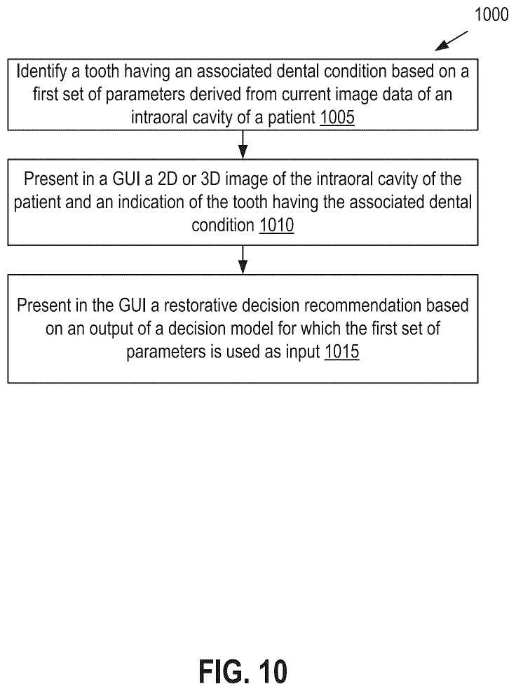

FIG. 10 illustrates a flow diagram for a method 1000 of presenting a restorative decision recommendation in a GUI of a dental diagnostics hub, in accordance with at least one embodiment of the present disclosure. At block 1005, processing logic identifies a tooth having an associated dental condition based on a first set of parameters derived from current image data of an intraoral cavity of a patient. In at least one embodiment, the current image data is received in the same or similar manner as described above with respect to block 905 of the method 900. In at least one embodiment, processing logic identifies the tooth having the associated dental condition by: (1) comparing the first set of parameters to a second set of parameters derived from prior image data of the intraoral cavity captured prior to the current image data; and (2) identifying the tooth by determining that a difference between a parameter from the first set of parameters and a parameter from the second set of parameters satisfies a threshold condition. For example, any time there is a new scan or CBCT of a patient, that information is stored in a record associated with that patient (e.g., in the past intraoral scan data 438). As new information becomes available, processing logic may compute or re-compute various parameters (e.g., decay volume proportion). If it is determined that a change in the computed/re-computed parameter crosses a threshold value for a given tooth, this tooth may be identified in the GUI as potentially having a condition. In at least one embodiment, the current image data corresponds to a first imaging modality, and the prior image data corresponds to a second imaging modality that is different from the first imaging modality (e.g., an advantage of the recommendation engine 433 being agnostic as to the type of modality used to derive parameters).

At block 1010, processing logic presents in a GUI a 2D or 3D image of the intraoral cavity of the patient and an indication of the tooth having the associated dental condition. For example, the indication may comprise one or more of a label on the tooth, an outline over the tooth, or a color of the tooth.

At block 1015, processing logic presents in the GUI a restorative decision recommendation based on an output of a decision model for which the first set of parameters is used as input. In at least one embodiment, the restorative decision recommendation is generated, for example, as described above with respect to the method 900. In at least one embodiment, the restorative decision recommendation is presented in the GUI responsive to a user selection of the tooth in the 2D or 3D image. The recommendation may also be provided visually next to an AOI associated with the tooth for which the restoration is recommended. For example, the doctor may review the restorative decision recommendation and associated AOI to make their own assessment as to the existence and/or severity of the dental condition. This may include zooming in or out on the AOIs, panning, rotating the view of the AOIs, looking at additional data regarding the AOIs such as NIRI imaging data, ultraviolet imaging data, color data, x-ray data, and so on.

FIG. 11 illustrates a flow diagram for a method 1100 of generating a restorative decision recommendation from parameters derived from image data by a trained machine learning model, in accordance with at least one embodiment of the present disclosure. At block 1105, processing logic receives image data corresponding to an intraoral cavity of a patient, which may be received in a similar manner as described above with respect to block 905 of the method 900.

At block 1110, processing logic applies a trained machine learning model to the image data to derive a plurality of parameters from the image data. In at least one embodiment, the trained machine learning model is adapted to compute or estimate a volume of restorative material present on or in a tooth in the image data, and wherein deriving the plurality of parameters comprises computing at least one of a restorative volume or a surface proportion from the estimated volume of restorative material. For example, the machine model may be trained to segment 2D projections of a 3D model of the patient's dentition (e.g., as discussed above with respect to FIGS. 5-7), from which the restorative volume or surface proportion may be computed. In at least one embodiment, the trained machine learning model is adapted to receive image data corresponding to different imaging modalities as input. In other embodiments, different machine learning models may be utilized, with each being adapted to segment images of different modalities. In at least one embodiment, the different imaging modalities are independently selected from an intraoral scan, a radiograph, or a cone-beam computed tomography (CBCT) scan.

At block 1115, processing logic applies a decision model to the plurality of parameters to generate a restorative decision recommendation. In at least one embodiment, the restorative decision recommendation is generated, for example, as described above with respect to the method 900.

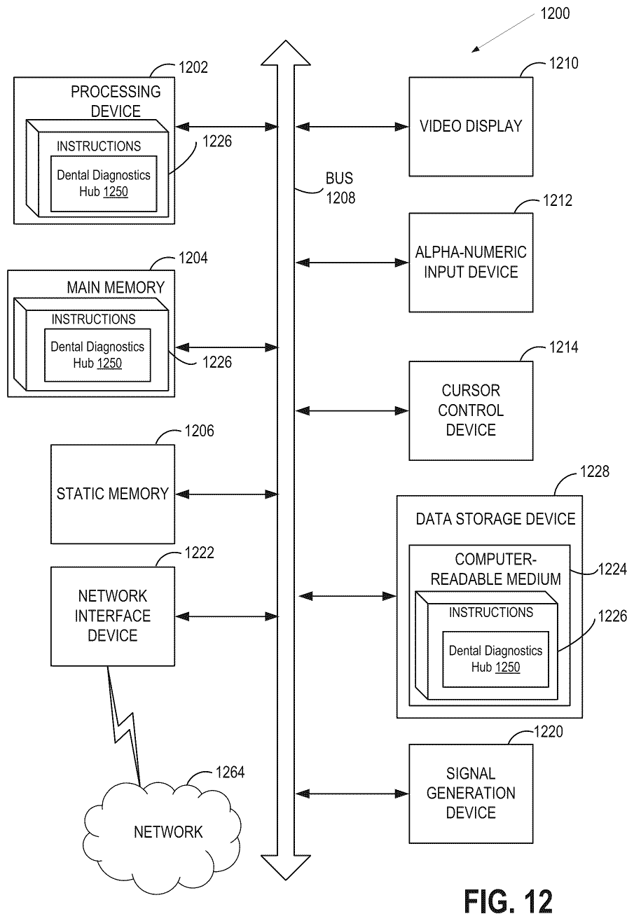

FIG. 12 illustrates a diagrammatic representation of a machine in the example form of a computing device 1200 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein. In at least one embodiment, the computing device 1200 corresponds to computing device 405 of FIG. 4.

The example computing device 1200 includes a processing device 1202, a main memory 1204 (e.g., read-only memory (ROM), flash memory, dynamic random access memory (DRAM) such as synchronous DRAM (SDRAM), etc.), a static memory 1206 (e.g., flash memory, static random access memory (SRAM), etc.), and a secondary memory (e.g., a data storage device 1228), which communicate with each other via a bus 1208.

Processing device 1202 represents one or more general-purpose processors such as a microprocessor, central processing unit, or the like. More particularly, the processing device 1202 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 1202 may also be one or more special-purpose processing devices such as an application specific integrated circuit (ASIC), a field programmable gate array (FPGA), a digital signal processor (DSP), network processor, or the like. Processing device 1202 is configured to execute the processing logic (instructions 1226) for performing operations and steps discussed herein.

The computing device 1200 may further include a network interface device 1222 for communicating with a network 1264. The computing device 1200 also may include a video display unit 1210 (e.g., a liquid crystal display (LCD) or a cathode ray tube (CRT)), an alphanumeric input device 1212 (e.g., a keyboard), a cursor control device 1214 (e.g., a mouse), and a signal generation device 1220 (e.g., a speaker).

The data storage device 1228 may include a machine-readable storage medium (or more specifically a non-transitory computer-readable storage medium) 1224 on which is stored one or more sets of instructions 1226 embodying any one or more of the methodologies or functions described herein. A non-transitory storage medium refers to a storage medium other than a carrier wave. The instructions 1226 may also reside, completely or at least partially, within the main memory 1204 and/or within the processing device 1202 during execution thereof by the computer device 1200, the main memory 1204 and the processing device 1202 also constituting computer-readable storage media.

The computer-readable storage medium 1224 may also be used to store a recommendation engine 1250, which may correspond to the similarly named component of FIG. 4. The computer readable storage medium 1224 may also store a software library containing methods for a recommendation engine 1250. While the computer-readable storage medium 1224 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any non-transitory medium (e.g., a medium other than a carrier wave) that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

The following exemplary embodiments are now described:

Embodiment 1: A method of providing restorative decision support for a dental patient, the method comprising: receiving image data of an intraoral cavity of a patient, the image data corresponding to one or more imaging modalities; deriving a plurality of parameters from the image data; applying a decision model to the plurality of parameters; and generating a restorative decision recommendation based on an output of the decision model.

Embodiment 2: The method of Embodiment 1, wherein the one or more imaging modalities comprises an imaging modality selected from an intraoral scan, a radiograph, or a cone-beam computed tomography (CBCT) scan.

Embodiment 3: The method of Embodiment 2, wherein the one or more imaging modalities comprises the intraoral scan, and wherein the image data comprises one or more three-dimensional (3D) point clouds and at least one of two-dimensional (2D) near infrared (NIR) images, 2D ultraviolet images, or 2D color images.

Embodiment 4: The method of Embodiment 2, wherein the one or more imaging modalities comprise the radiograph, and wherein the image data comprises one or more of a panoramic radiograph, a bitewing radiograph, or a periapical radiograph.

Embodiment 5: The method of Embodiment 2, wherein the image data corresponds to two or more of the imaging modalities.

Embodiment 6: The method of any of the preceding Embodiments, wherein the plurality of parameters are each selected from a geometric parameter, a volume/area parameter, or a fracture classification parameter.

Embodiment 7: The method of Embodiment 6, wherein the geometric parameter comprises an inter-cuspal width.

Embodiment 8: The method of Embodiment 6, wherein the volume/area parameter comprises one or more of a restorative volume proportion, a decay volume proportion, or a restorative surface proportion.

Embodiment 9: The method of Embodiment 6, wherein the fracture classification parameter comprises information descriptive of a tooth fracture location and a tooth fracture depth.

Embodiment 10: The method of any of the preceding Embodiments, wherein the decision model comprises one or more of a decision tree or a neural network.

Embodiment 11: The method of any of the preceding Embodiments, wherein the restorative decision recommendation comprises one or more of: a direct restoration recommendation or an indirect restoration recommendation, or an indication of a dental condition and a severity level for the dental condition.

Embodiment 12: The method of Embodiment 11, wherein the dental condition is selected from a group consisting of caries, gum recession, tooth wear, malocclusion, tooth crowding, tooth spacing, plaque, tooth stains, tooth cracks, cervical defects, and chipped or broken teeth.

Embodiment 13: The method of any of the preceding Embodiments, further comprising: presenting the restorative decision recommendation for display in a graphical user interface (GUI).

Embodiment 14: The method of any of the preceding Embodiments, further comprising: storing, in a record associated with a dentist to whom the restorative decision recommendation was provided, the restorative decision recommendation and an actual restorative decision made by the dentist in a key performance indicator (KPI) database.

Embodiment 15: A method comprising: identifying a tooth having an associated dental condition based on a first set of parameters derived from current image data of an intraoral cavity of a patient; presenting in a graphical user interface (GUI) a 2D or 3D image of the intraoral cavity of the patient and an indication of the tooth having the associated dental condition; and presenting in the GUI a restorative decision recommendation based on an output of a decision model for which the first set of parameters is used as input.

Embodiment 16: The method of Embodiment 15, wherein the restorative decision recommendation is presented in the GUI responsive to a user selection of the tooth in the 2D or 3D image, and wherein the indication comprises one or more of a label on the tooth, an outline over the tooth, or a color of the tooth.

Embodiment 17: The method of either Embodiment 15 or Embodiment 16, wherein identifying the tooth having the associated dental condition comprises: comparing the first set of parameters to a second set of parameters derived from prior image data of the intraoral cavity captured prior to the current image data; and identifying the tooth by determining that a difference between a parameter from the first set of parameters and a parameter from the second set of parameters satisfies a threshold condition.

Embodiment 18: The method of Embodiment 17, wherein the current image data corresponds to a first imaging modality, and wherein the prior image data corresponds to a second imaging modality that is different from the first imaging modality.

Embodiment 19: A method comprising: receiving image data corresponding to an intraoral cavity of a patient; applying a trained machine learning model to the image data to derive a plurality of parameters from the image data; and applying a decision model to the plurality of parameters to generate a restorative decision recommendation.

Embodiment 20: The method of Embodiment 19, wherein the trained machine learning model is adapted to compute or estimate a volume of restorative material present on or in a tooth in the image data, and wherein deriving the plurality of parameters comprises computing at least one of a restorative volume or a surface proportion from the estimated volume of restorative material.

Embodiment 21: The method of either Embodiment 19 or Embodiment 20, wherein the trained machine learning model is adapted to receive image data corresponding to different imaging modalities as input, and wherein the different imaging modalities are independently selected from an intraoral scan, a radiograph, or a cone-beam computed tomography (CBCT) scan.

Embodiment 22: A dental diagnostics system comprising: a memory; and a processing device to execute instructions from the memory to perform a method comprising: receiving image data of an intraoral cavity of a patient, the image data corresponding to one or more imaging modalities; deriving a plurality of parameters from the image data; applying a decision model to the plurality of parameters; and generating a restorative decision recommendation based on an output of the decision model.

Embodiment 23: An intraoral scanning system comprising: an intraoral scanner; and the dental diagnostics system of Embodiment 22.

Embodiment 24: A non-transitory computer-readable storage medium having instructions encoded thereon that, when executed by a computing device, cause the computing device to perform the method of any of Embodiments 1-21.

Claim language or other language herein reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

It is to be understood that the above description is intended to be illustrative, and not restrictive. Many other embodiments will be apparent upon reading and understanding the above description. Although embodiments of the present disclosure have been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

What is claimed is:

1. A method of providing restorative decision support for a dental patient, the method comprising:
receiving image data of an intraoral cavity of a patient, the image data corresponding to one or more imaging modalities;
deriving a plurality of parameters from the image data;
applying a decision model to the plurality of parameters; and
generating a restorative decision recommendation based on an output of the decision model.

2. The method of claim 1, wherein the one or more imaging modalities comprises an imaging modality selected from an intraoral scan, a radiograph, or a cone-beam computed tomography (CBCT) scan.

3. The method of claim 2, wherein the one or more imaging modalities comprises the intraoral scan, and wherein the image data comprises one or more three-dimensional (3D) point clouds and at least one of two-dimensional (2D) near infrared (NIR) images, 2D ultraviolet images, or 2D color images.

4. The method of claim 2, wherein the one or more imaging modalities comprise the radiograph, and wherein the image data comprises one or more of a panoramic radiograph, a bitewing radiograph, or a periapical radiograph.

5. The method of claim 2, wherein the image data corresponds to two or more of the imaging modalities.

6. The method of claim 1, wherein the plurality of parameters are each selected from a geometric parameter, a volume/area parameter, or a fracture classification parameter.

7. The method of claim 6, wherein the geometric parameter comprises an inter-cuspal width.

8. The method of claim 6, wherein the volume/area parameter comprises one or more of a restorative volume proportion, a decay volume proportion, or a restorative surface proportion.

9. The method of claim 6, wherein the fracture classification parameter comprises information descriptive of a tooth fracture location and a tooth fracture depth.

10. The method of claim 1, wherein the decision model comprises one or more of a decision tree or a neural network.

11. The method of claim 1, wherein the restorative decision recommendation comprises one or more of:
a direct restoration recommendation or an indirect restoration recommendation, or an indication of a dental condition and a severity level for the dental condition.

12. The method of claim 11, wherein the dental condition is selected from a group consisting of caries, gum recession, tooth wear, malocclusion, tooth crowding, tooth spacing, plaque, tooth stains, tooth cracks, cervical defects, and chipped or broken teeth.

13. The method of claim 1, further comprising:
presenting the restorative decision recommendation for display in a graphical user interface (GUI).

14. The method of claim 1, further comprising:
storing, in a record associated with a dentist to whom the restorative decision recommendation was provided, the restorative decision recommendation and an actual restorative decision made by the dentist in a key performance indicator (KPI) database.

15. A method comprising:
identifying a tooth having an associated dental condition based on a first set of parameters derived from current image data of an intraoral cavity of a patient;
presenting in a graphical user interface (GUI) a 2D or 3D image of the intraoral cavity of the patient and an indication of the tooth having the associated dental condition; and
presenting in the GUI a restorative decision recommendation based on an output of a decision model for which the first set of parameters is used as input.

16. The method of claim 15, wherein the restorative decision recommendation is presented in the GUI responsive to a user selection of the tooth in the 2D or 3D image, and wherein the indication comprises one or more of a label on the tooth, an outline over the tooth, or a color of the tooth.

17. The method of claim 15, wherein identifying the tooth having the associated dental condition comprises:
comparing the first set of parameters to a second set of parameters derived from prior image data of the intraoral cavity captured prior to the current image data; and
identifying the tooth by determining that a difference between a parameter from the first set of parameters and a parameter from the second set of parameters satisfies a threshold condition.

18. The method of claim 17, wherein the current image data corresponds to a first imaging modality, and wherein the prior image data corresponds to a second imaging modality that is different from the first imaging modality.

19. A method comprising:
receiving image data corresponding to an intraoral cavity of a patient;
applying a trained machine learning model to the image data to derive a plurality of parameters from the image data; and
applying a decision model to the plurality of parameters to generate a restorative decision recommendation.

20. The method of claim 19, wherein the trained machine learning model is adapted to compute or estimate a volume of restorative material present on or in a tooth in the image data, and wherein deriving the plurality of parameters comprises computing at least one of a restorative volume or a surface proportion from the estimated volume of restorative material.

21. The method of claim 19, wherein the trained machine learning model is adapted to receive image data corresponding to different imaging modalities as input, and wherein the different imaging modalities are independently selected from an intraoral scan, a radiograph, or a cone-beam computed tomography (CBCT) scan.

22. A dental diagnostics system comprising:
a memory; and
a processing device to execute instructions from the memory to perform a method comprising:
receiving image data of an intraoral cavity of a patient, the image data corresponding to one or more imaging modalities;
deriving a plurality of parameters from the image data;
applying a decision model to the plurality of parameters; and
generating a restorative decision recommendation based on an output of the decision model.

23. An intraoral scanning system comprising:
an intraoral scanner; and
the dental diagnostics system of claim 22.

24. A non-transitory computer-readable storage medium having instructions encoded thereon that, when executed by a computing device, cause the computing device to perform the method of claim 1.
